(19) United States
(12) Patent Application Publication
(10) Pub. No.: US 2024/0277781 A1
(43) Pub. Date: Aug. 22, 2024

(54) Compositions Comprising Microbes and Methods of Use and Making Thereof

(71) Applicant: Pendulum Therapeutics, Inc., San Francisco, CA (US)

(72) Inventors: John S. Eid, San Francisco, CA (US); Paul J. McMurdie, Fremont, CA (US); Orville G. Kolterman, La Jolla, CA (US)

(21) Appl. No.: 18/569,805

(22) PCT Filed: Jun. 21, 2022
(86) PCT No.: PCT/US22/34381

§ 371 Date: Dec. 13, 2023

Related U.S. Application Data
(60) Provisional application No. 63/212,794, filed on Jun. 21, 2021.

(51) Int. Cl. A61K 35/742; A61K 9/00; A61K 35/00; A61K 35/741; A61K 35/745; A61K 45/06; A61P 1/16

(52) U.S. Cl. CPC A61K 35/742; A61K 9/0053; A61K 35/741; A61K 35/745; A61K 45/06; A61P 1/16; A61K 2035/115

(57) ABSTRACT

Provided are methods of increasing ursodeoxycholic acid (UDCA) levels in a mammalian subject. The methods comprise orally administering to the mammalian subject a composition comprising one or more bacterial strains in an amount effective to increase UDCA levels in the mammalian subject. Also provided are methods of antagonizing farnesoid X receptor (FXR) in intestinal cells of a mammalian subject. Such methods comprise orally administering to the mammalian subject a composition comprising one or more bacterial strains in an amount effective to antagonize FXR in intestinal cells of the mammalian subject. In certain embodiments, the mammalian subject has diabetes, e.g., type 2 diabetes. According to some embodiments, the mammalian subject suffers from a liver disorder, obesity, or both. In certain embodiments, the mammalian subject has colorectal cancer. According to some embodiments, the mammalian subject has gallstones.

### CROSS-REFERENCE TO RELATED APPLICATIONS

This application claims the benefit of U.S. Provisional Patent Application No. 63/212,794, filed Jun. 21, 2021, which application is incorporated herein by reference in its entirety.

### BACKGROUND

The body of an individual is inhabited by trillions of microbes across various locations, often referred to as microbiomes. Microbiomes can play a key role in many health conditions and diseases. Despite the interrelation between microbiomes and health, the complexity of the various microbiomes, as well as difficulties in characterizing, categorizing, and analyzing microbiome constituents has made understanding microbiomes challenging. Consequently, these challenges have presented hurdles in the development of diagnostic and therapeutic applications for microbiome-related health conditions and diseases. The present disclosure provides methods, systems, compositions, and kits to address the need for microbiome-related treatment of health conditions and disease.

34.2 million Americans were estimated to have diabetes in 2018—over one in ten—with an estimated annual cost burden of $327 billion and growing, due to an estimated 1.5 million new diagnoses annually. Approximately 90% of total diabetes diagnoses are type 2 (T2D). While genetic factors affect susceptibility, it is increasingly evident that Western lifestyle and diet plays a large role in T2D pathogenesis, as do resident gut microbiota. The therapeutic effects of T2D drugs are at least partially mediated by gut microbes, including widely used metformin, and many T2D drugs have been shown to alter the gut microbiome. The gut microbiome contributes significantly to predictions of glycemic response and alterations in the gut microbiome induced by lifestyle are mechanistically implicated in aspects of disease progression. Even positive metabolic outcomes from calorie restriction appear to depend on the preintervention state of the gut microbiome, and certain genera in the healthy human gut are known to be underrepresented in subjects with T2D. Metagenomic surveys have shown that these microbiome alterations often result in a reduction in the capacity or resiliency of short chain fatty acid production in the gut microbiome, especially butyrate.

Dietary fibers, and other dietary oligomers that escape digestion in the upper human gastrointestinal tract, are hydrolyzed and fermented by the microbiota of the lower gut, releasing ‘short-chain fatty acids’ (SCFA): 95% of typical SCFA content are the 2C-4C forms, respectively acetate, propionate, and butyrate. SCFAs are among the most important bacterial metabolites yet identified, serving as catabolic substrates for host cell oxidation—notably butyrate is the primary energy substrate of colonocytes—as well as direct activation of G-coupled receptors and inhibition of histone deacetylases. SCFAs can stimulate the proliferation, differentiation and function of Tregs, as well as increase the production of several cytokines (e.g., IL-10) and hence participate in maintaining the balance between pro- and anti-inflammatory immune pathways. SCFAs can enter hepatic or systemic circulation, albeit with molecule-specific transport and fate, where they appear to directly affect metabolism or peripheral tissue function. Robust gut SCFA production has been associated with a reduced risk for certain conditions, including irritable bowel syndrome, inflammatory bowel diseases, cardiovascular disease, and cancer—particularly colorectal cancer. Regarding cardiometabolic health, SCFAs are proposed to beneficially modulate glucose homeostasis and insulin sensitivity via adipose tissue, skeletal muscle, and liver tissue functions. However, there remains a need to fully establish these mechanisms in well-controlled human intervention studies.

A known limitation of fecal SCFA measurements is that a variable and overwhelming fraction (90-95%) of luminally-produced SCFA are absorbed prior to excretion in the feces, and that circulating SCFA are more predictive for metabolic indicators such as insulin sensitivity, lipolysis, and glucagon-like peptide-1 (GLP-1) concentrations.

Some of the aforementioned indicators are also modulated by microbiota-dependent changes to the bile acid pool. Bile acids are critical to dietary absorption of lipids and fat-soluble vitamins, and also regulate numerous host metabolic pathways. Human primary bile acids, cholic acid (CA) and chenodeoxycholic acid (CDCA), themselves implicated in metabolic signaling, are synthesized by the liver from cholesterol and conjugated with taurine or glycine by hepatocytes en route to mixing in the gallbladder. Conjugated bile acids are efficiently (˜95%) reabsorbed in the distal ileum (‘enterohepatic circulation’) following catalysis by gut microbiota of highly specific epimerization and redox reactions to produce secondary bile acids that alter the composition, solubility, and signaling properties of the bile acid pool. The bile acid receptors, farnesoid X receptor (FXR)] and a G-protein-coupled receptor (TGR5), have elicited considerable interest as targets in metabolic diseases, and there is now extensive support for microbiota-dependent modulation of these receptors via modification of bile acids.

### SUMMARY

In embodiments, disclosed herein are compositions comprising one or more microbes selected from the group consisting of Akkermansia sp., Clostridium sp., Bifidobacterium sp., Anerobutyricum sp. and Eubacterium sp.

In embodiments, disclosed herein are compositions comprising microbes from 2 or more, 3 or more of the group consisting of Akkermansia sp., Bifidobacterium sp., Clostridium sp., Anerobutyricum sp.

In embodiments, disclosed herein are compositions comprising microbes selected from 2 or more, 3 or more, four or more of the group consisting of Akkermansia sp., Clostridium sp., Bifidobacterium sp., and Eubacterium sp.

In embodiments, disclosed herein are compositions comprising Clostridium sp.

In embodiments, disclosed herein are compositions comprising one or more microbes having a 16S rRNA sequence comprising at least 95% identity to the full length of a 16S rRNA sequence of a microbe selected from the group consisting of Akkermansia muciniphila ATCC BAA-835, Bifidobacterium infantis ATCC 15697, Clostridium butyricum DSM 10702, Clostridium beijerinckii NCIMB 8052, and Anerobutyricum hallii DSM 3353.

In embodiments, disclosed herein are compositions comprising microbes having 16S rRNA sequence comprising at least 95% identity to the full length of a 16S rRNA sequence of a microbe from 2 or more, 3 or more, four or more, five or more, of the group consisting of Akkermansia muciniphila ATCC BAA-835, Bifidobacterium infantis ATCC 15697, Clostridium butyricum DSM 10702, Clostridium beijerinckii NCIMB 8052, and Anerobutyricum hallii DSM 3353.

In embodiments, disclosed herein are compositions comprising microbes having 16S rRNA sequence comprising at least 97% identity to the full length of a 16S rRNA sequence of a microbe from 2 or more, 3 or more, four or more, or all five of the group consisting of Bifidobacterium infantis ATCC 15697, Akkermansia muciniphila ATCC BAA-835, Clostridium butyricum DSM 10702, Anerobutyricum hallii DSM 3353, Clostridium beijerinckii NCIMB 8052.

In embodiments, disclosed herein are compositions comprising microbes having 16S rRNA sequence comprising at least 98% identity to the full length of a 16S rRNA sequence of a microbe from 2 or more, 3 or more, four or more, or all five of the group consisting of Bifidobacterium infantis ATCC 15697, Akkermansia muciniphila ATCC BAA-835, Clostridium butyricum DSM 10702, Anerobutyricum hallii DSM 3353, Clostridium beijerinckii NCIMB 8052.

In embodiments, disclosed herein are compositions comprising one or more microbes selected from the group consisting of Akkermansia muciniphila, Clostridium butyricum, Clostridium beijerinckii, Bifidobacterium infantis, and Anerobutyricum hallii.

In embodiments, disclosed herein are compositions comprising-Akkermansia muciniphila, Clostridium butyricum, Clostridium beijerinckii, Bifidobacterium infantis, and Anerobutyricum hallii.

In embodiments, disclosed herein are compositions comprising microbes from 2 or more, 3 or more, four or more, or all five of the group consisting of Akkermansia muciniphila ATCC BAA-835, Bifidobacterium infantis ATCC 15697, Clostridium butyricum DSM 10702, Clostridium beijerinckii NCIMB 8052, and Anerobutyricum hallii DSM 3353.

In embodiments, disclosed herein are compositions comprising Akkermansia muciniphila ATCC BAA-835, Bifidobacterium infantis ATCC 15697, Clostridium butyricum DSM 10702, Clostridium beijerinckii NCIMB 8052, and Anerobutyricum hallii DSM 3353.

In embodiments, disclosed herein are compositions comprising Clostridium butyricum.

In embodiments, disclosed herein are compositions comprising Akkermansia muciniphila, Anerobutyricum hallii, and Clostridium butyricum.

In embodiments, disclosed herein are compositions comprising Akkermansia muciniphila ATCC BAA-835, Anerobutyricum hallii DSM 3353, and Clostridium butyricum DSM 10702.

In embodiments, disclosed herein are compositions comprising microbes from 2 or more, 3 or more, four or more, or all five of the group consisting of Bifidobacterium infantis, Akkermansia muciniphila, Clostridium butyricum, Anerobutyricum hallii, Clostridium beijerinckii.

Certain embodiments include a composition or a composition of any of the preceding embodiments comprising at least one effective protein component extracted from at least one of the microbes selected from the group consisting of Bifidobacterium infantis, Akkermansia muciniphila, Clostridium butyricum, Anerobutyricum hallii, Clostridium beijerinckii.

Certain embodiments include a composition of any of the preceding embodiments, wherein the formulation further comprises one or more additional microbe strains having a 16S rRNA sequence comprising at least 95% identity to the full length of a 16S rRNA sequence of a microbe selected from the group consisting of Anaerostipes caccae, Bacteroides finegoldii, Bacteroides ovatus, Bacteroides stercoris, Bifidobacterium bifidum, Bifidobacterium longum, Blautia hydrogenotrophica, Blautia producta, Butyrivibrio fibrisolvens, Clostridium acetobutylicum, Clostridium aminophilum, Clostridium colinum, Clostridium indolis, Clostridium innocuum, Clostridium orbiscindens, Enterococcus faecium, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faeccis, Roseburia hominis, Roseburia intestinalis, Collinsella aerofaciens, Coprococcus comes, Eubacterium limosum, and Ruminococcus faecis, and all combinations thereof.

Certain embodiments include a composition of any of the preceding embodiments, wherein the formulation further comprises one or more additional microbe strains having a 16S rRNA sequence comprising at least 97% identity to the full length of a 16S rRNA sequence of a microbe selected from the group consisting of: Anaerostipes caccae, Bacteroides finegoldii, Bacteroides ovatus, Bacteroides stercoris, Bifidobacterium bifidum, Bifidobacterium longum, Blautia hydrogenotrophica, Blautia producta, Butyrivibrio fibrisolvens, Clostridium acetobutylicum, Clostridium aminophilum, Clostridium colinum, Clostridium indolis, Clostridium innocuum, Clostridium orbiscindens, Enterococcus faecium, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faeccis, Roseburia hominis, Roseburia intestinalis, Collinsella aerofaciens, Coprococcus comes, Eubacterium limosum, and Ruminococcus faecis, and all combinations thereof.

Certain embodiments include a composition or a composition of any of the preceding embodiments comprising at least one effective protein component extracted from at least one of the microbes selected from the group consisting of Anaerostipes caccae, Bacteroides finegoldii, Bacteroides ovatus, Bacteroides stercoris, Bifidobacterium bifidum, Bifidobacterium infantis, Bifidobacterium longum, Blautia hydrogenotrophica, Blautia producta, Butyrivibrio fibrisolvens, Clostridium acetobutylicum, Clostridium aminophilum, Clostridium colinum, Clostridium indolis, Clostridium innocuum, Clostridium orbiscindens, Enterococcus faecium, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faeccis, Roseburia hominis, Roseburia intestinalis, Collinsella aerofaciens, Coprococcus comes, Eubacterium limosum, and Ruminococcus faecis, and all combinations thereof.

A composition comprising at least one microbe that produces a secondary bile acid from a primary bile acid. The secondary bile acid is any one of ursodeoxycholic acid (UDCA) or glyco-ursodeoxycholic (G-UDCA) acid and the primary bile acid is chenodeoxycholic acid (CDCA).

A composition comprising at least one microbe that expresses the enzymes 7α- and 7β-hydroxysteroid dehydrogenase is also disclosed. These enzymes produced by the microbes convert a primary bile acid (e.g., CA, CDCA) into a secondary bile acid (e.g., UDCA or G-UDCA, tauro-UDCA, and others FIG. 3b). In certain embodiments, the microbe, in the composition, is a bacteria chosen from the genus Clostridia. In one embodiment, the bacteria is chosen from Clostridium butyricum.

In certain embodiments, a composition is herein disclosed comprising a combination of a first group of microbes and a second group of microbes, wherein the first group of microbes produces butyrate and the second group of microbes converts a primary bile acid or salt thereof to a secondary bile acid or salt thereof. Without being bound to a particular theory, it is proposed that the butyrate and the secondary bile acid, in combination, increase GLP-1 production in L-cells as compared to the increase due to either butyrate or the secondary bile acid alone. In particular embodiments, the combination of butyrate and secondary bile acid (e.g., UDCA) result in a synergistic increase in GLP-1 production as compared to what would be expected from the addition of the effects of butyrate and secondary bile acid alone.

A composition comprising a first group of one or more microbes that produces an intermediate molecule from a prebiotic, wherein the intermediate molecule is any one or more of acetate, lactate or glucose, a second group of one or more microbes that uses the intermediate molecule to produce butyrate, and a third group of one or more microbes that produces a UDCA from a primary bile acid wherein the butyrate and the UDCA synergistically increase GLP-1 production by targeting intestinal cells.

Certain embodiments include a composition of any of the preceding embodiments, wherein the formulation increases GLP-1 production in the L-cells of a subject treated with the formulation.

Certain embodiments include a composition of any of the preceding embodiments, wherein the formulation decreases blood sugar levels in a subject treated with the composition.

Certain embodiments include a composition of any of the preceding embodiments, wherein the formulation dissolves gallstones in a subject treated with the composition.

Certain embodiments include a composition of any of the preceding embodiments, wherein the formulation reduces the size of gallbladder stones in a subject treated with the composition.

Certain embodiments include a composition of any of the preceding embodiments, wherein the formulation reduces discomfort due to biliary distention in a subject treated with the composition.

Certain embodiments include a composition of any of the preceding embodiments, wherein the formulation increases UDCA levels in a subject that effectively antagonizes the Farnesoid X Receptor (FXR) in the subject treated with the composition.

Certain embodiments include a composition of any of the preceding embodiments, wherein the subject suffers from liver inflammation, biliary disorders, uncontrolled blood sugar spikes, osteoporosis, cardiovascular disorders, or FXR-dependent cancers, non-FXR dependent.

Certain embodiments include a composition of any of the preceding embodiments, wherein the subject is a mammal. In particular embodiments, the mammal is a human.

Certain embodiments include a composition of any of the preceding embodiments, wherein the formulation further comprises an enteric coating.

Certain embodiments include a composition of any of the preceding embodiments, wherein the composition is formulated as an enteric-coated pill. In some aspects, the method may comprise formulating the composition as an enteric-coated pill, wherein the enteric-coating is formed by a pH sensitive polymer. In some aspects, the method may comprise formulating the composition as an enteric-coated pill, wherein the enteric-coating is formed by a pH sensitive polymer, wherein the polymer is eudragit FS30D.

Certain embodiments include a composition of any of the preceding embodiments, wherein the formulation further comprises an effective amount of a preservative.

Certain embodiments include a composition of any of the preceding embodiments, wherein the formulation further comprises a prebiotic.

Certain embodiments include a composition of any of the preceding embodiments, wherein the formulation further comprises an enteric coating.

Certain embodiments include a composition of any of the preceding embodiments, wherein the formulation further comprises a prebiotic selected from the group consisting of inulin, green banana, Reishi, tapioca, oats, pectin, potato or extracts thereof, complex carbohydrates, complex sugars, resistant dextrins, resistant starch, amino acids, peptides, nutritional compounds, biotin, polydextrose, fructooligosaccharide (FOS), galactooligosaccharides (GOS), starch, lignin, psyllium, chitin, chitosan, gums (e.g. guar gum), high amylose cornstarch (HAS), cellulose, β-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), and any combination thereof.

Certain embodiments include a composition of any of the preceding embodiments, wherein at least one of the microbes is lyophilized.

Certain embodiments include a composition of any of the preceding embodiments, wherein at least one of the microbes is viable.

Certain embodiments include a composition of any of the preceding embodiments, wherein at least one of the microbes is non-viable.

Certain embodiments include a composition of any of the preceding embodiments, wherein at least one of the microbes has been pasteurized.

Certain embodiments include a composition of any of the preceding embodiments, wherein the at least about 95% sequence identity is selected from the group consisting of: at least about 96%, at least about 97%, at least about 98%, at least about 99%, at least about 99.5%, and at least about 99.5% sequence identity to a rRNA sequence.

Certain embodiments include a composition of any of the preceding embodiments, wherein the pharmaceutical composition is substantially free of fecal matter obtained from a subject.

Certain embodiments include a composition of any of the preceding embodiments, wherein the at least one of the microbes comprises a population of the microbes.

Certain embodiments include a composition of any of the preceding embodiments, wherein the composition is formulated for oral delivery.

Certain embodiments include a composition of any of the preceding embodiments, wherein the composition is formulated as a nutritional supplement.

Certain embodiments include a composition of any of the preceding embodiments, wherein the composition is formulated as a medical food.

Certain embodiments include a composition of any of the preceding embodiments, wherein the composition is formulated as a medical probiotic.

Certain embodiments include a composition of any of the preceding embodiments, wherein the composition is formulated for dietary management of a gut disorder.

Certain embodiments include a composition of any of the preceding embodiments, wherein the composition is formulated for dietary management of Inflammatory bowel syndrome (IBS).

Certain embodiments include a composition of any of the preceding embodiments, wherein the composition is formulated for anal delivery.

Certain embodiments include a composition of any of the preceding embodiments, wherein the composition is formulated as a pill.

Certain embodiments include a composition of any of the preceding embodiments, wherein the composition is formulated as a capsule.

Certain embodiments include a composition of any of the preceding embodiments, wherein the composition is formulated in a liquid form suitable for administration via an enema.

Certain embodiments include a composition of any of the preceding embodiments, wherein the composition is formulated as a suppository.

Certain embodiments include a composition of any of the preceding embodiments, wherein the composition is formulated in a liquid form suitable for delivery via injection.

A method of producing the microbes of any of the preceding embodiments, the method comprising genetically-modifying the microbes to generate recombinant microbes. In some aspects, the method may comprise genetically-modifying the microbes to generate recombinant microbes, wherein an operon controls growth of the recombinant microbe.

Certain embodiments include a composition of any of the preceding embodiments, wherein the composition is formulated for delivery of the microbes to the subject's ileum region.

Certain embodiments include a composition of any of the preceding embodiments, wherein the composition is formulated for delivery of the microbes to the subject's liver region.

Certain embodiments include a composition of any of the preceding embodiments, wherein the composition is formulated for delivery of the microbes into the gallbladder region.

Certain embodiments include a composition of any of the preceding embodiments, wherein the composition is formulated for delivery of the microbes to the subject's colon region.

Certain embodiments include a composition of any of the preceding embodiments, wherein the composition is formulated for delivery of the microbes to the subject's ileum and colon region.

Certain embodiments include a composition of any of the preceding embodiments, wherein the microbes comprise a synergistic stability in the composition as compared to individual strains.

In some embodiments, disclosed herein is a method of treating a subject with at least one of the compositions of any of the preceding embodiments.

In some embodiments, disclosed herein is a method of reducing the size of the gallstones in the subject comprising administering to the subject at least one of the compositions of any of the preceding embodiments.

In some embodiments, disclosed herein is a method of reducing pain in response to gallstones in a subject comprising administering to the subject at least one of the compositions of any of the preceding embodiments.

In particular embodiments, the subject suffers from irritable bowel syndrome, inflammatory bowel disease, ulcerative colitis, diarrhea, constipation, leaky intestine, and/or Crohn's disease.

Embodiments include the methods of any of the preceding embodiments, wherein treating and/or administering results in a subject with an altered microbiome.

Embodiments include the methods of the preceding embodiments, wherein treating and/or administering results in a subject with an altered gut microbiome.

Embodiments include the methods of the preceding embodiments, wherein the composition is co-administered with an antibiotic.

Embodiments include the methods of the preceding embodiments, wherein the composition is administered after an antibiotic. In some aspects, the method may comprise of administering the composition after an antibiotic, wherein the composition is administered at least one hour after an antibiotic. In some aspects, the method may comprise of administering the composition after an antibiotic, wherein the composition is administered at least 2 hours after an antibiotic. In some aspects, the method may comprise of administering the composition after an antibiotic, wherein the composition is administered at least 12 hours after an antibiotic. In some aspects, the method may comprise of administering the composition after an antibiotic, wherein the composition is administered at least 1 day after an antibiotic. In some aspects, the method may comprise of administering the composition after an antibiotic, wherein the composition is administered at least 1 week after an antibiotic. In some aspects, the method may comprise of administering the composition after an antibiotic, wherein the composition is administered at least 2 weeks after an antibiotic.

Embodiments include the methods of the preceding embodiments, wherein the composition is administered after completion of an antibiotic regimen by the subject.

Embodiments include the methods of the preceding embodiments, wherein the composition is formulated as a dietary supplement.

Embodiments include the methods of the preceding embodiments, wherein the composition is formulated as a nutritional supplement.

Embodiments include the methods of the preceding embodiments, wherein the composition is formulated as a medical food.

Embodiments include the methods of the preceding embodiments, wherein the composition is formulated as a medical probiotic.

Embodiments include the methods of the preceding embodiments, wherein the composition is a biologic product.

Embodiments include the methods of the preceding embodiments, further comprising determining the sequence of a population of the subject's microbiome by sequencing. In some aspects, the method may further comprise determining the sequence of the subject's microbiome by sequencing, the sequencing comprises sequencing the 16S rRNA. In some aspects, the method may further comprise determining the sequence of the subject's microbiome by sequencing, the sequencing comprises sequencing the 23S rRNA. In some aspects, the method may further comprise determining the sequence of the subject's microbiome by sequencing, the sequencing comprises sequencing the 23S and 16S rRNA. In some aspects, the method may further comprise determining the sequence of the subject's microbiome by sequencing, the sequencing comprises Complete Biome Test resolution. In some aspects, the sequencing comprises long-read sequencing. In some aspects, the method may further comprise determining the sequence of the subject's microbiome by sequencing, wherein the determining the sequence of the population of the subject's microbiome is performed before treating the subject with the composition. In some aspects, the method may further comprise determining the sequence of the subject's microbiome by sequencing, wherein the determining the sequence of the population of the subject's microbiome is performed after treating the subject with the composition.

Embodiments include the methods of the preceding embodiments, further comprising transmitting data via machine-readable code.

Embodiments include the methods of the preceding embodiments, further comprising computing data via machine-readable code.

Embodiments include the methods of the preceding embodiments, further comprising storing data via machine-readable code.

Embodiments include the methods of the preceding embodiments, wherein the method further comprises a companion diagnostic.

Embodiments include the methods of the preceding embodiments, wherein the composition is delivered to the subject's ileum region.

Embodiments include the methods of the preceding embodiments, wherein the composition is delivered to the subject's colon region.

Embodiments include the methods of the preceding embodiments, wherein the composition is delivered to the subject's ileum and colon region.

Embodiments include the methods of the preceding embodiments, wherein the composition is administered before food intake. In some aspects, the method may comprise administering the composition before food intake, wherein the composition is administered at least one hour before food intake. In some aspects, the method may comprise of administering the composition before food intake, wherein the composition is administered at least 2 hours before food intake. In some aspects, the method may comprise administering the composition before food intake, wherein the composition is administered at least 3 hours before food intake. In some aspects, the method may comprise administering the composition before food intake, wherein the composition is administered at least 4 hours before food intake.

Embodiments include the methods of the preceding embodiments, wherein the microbes are administered with food intake.

### SEQUENCE INFORMATION

Nucleotide sequence information (including 16S rRNA sequence information) for microbial strain Akkermansia muciniphila, culture collection ATCC BAA-835, is available at International Nucleotide Sequence Database Collaboration (DDBJ/EMBL/GENBANK) accession number CP001071.1, which is herein incorporated by reference in its entirety.

Nucleotide sequence information (including 16S rRNA sequence information) for microbial strain Anaerofustis stercorihominis, culture collection DSM 17244, is available at DDBJ/EMBL/GENBANK accession number AJ518871.2, which is herein incorporated by reference in its entirety.

Nucleotide sequence information (including 16S rRNA sequence information) for microbial strain Anaerostipes caccae, culture collection DSM 14662, is available at DDBJ/EMBL/GENBANK accession number DS499744.1, which is herein incorporated by reference in its entirety.

Nucleotide sequence information (including 16S rRNA sequence information) for microbial strain Anaerostipes caccae, butyrate-producing bacterium L1-92, is available at DDBJ/EMBL/GENBANK accession number AJ270487.2, which is herein incorporated by reference in its entirety.

Nucleotide sequence information (including 16S rRNA sequence information) for microbial strain Anaerostipes hadrus, butyrate-producing bacterium SS2/1, is available at DDBJ/EMBL/GENBANK accession number AY305319.1, which is herein incorporated by reference in its entirety.

Nucleotide sequence information (including 16S rRNA sequence information) for microbial strain Anaerotruncus colihominis, culture collection DSM 17241, is available at DDBJ/EMBL/GENBANK accession number AJ315980.1, which is herein incorporated by reference in its entirety.

Nucleotide sequence information (including 16S rRNA sequence information) for microbial strain Bacteroides finegoldii, culture collection DSM 17565, is available at DDBJ/EMBL/GENBANK accession number GCA_000156195.1, which is herein incorporated by reference in its entirety.

Nucleotide sequence information (including 16S rRNA sequence information) for microbial strain Bacteroides ovatus, culture collection ATCC 8483, is available at DDBJ/EMBL/GENBANK accession number GCF_000154125.1, which is herein incorporated by reference in its entirety.

Nucleotide sequence information (including 16S rRNA sequence information) for microbial strain Bacteroides stercoris, culture collection ATCC 43183, is available at DDBJ/EMBL/GENBANK accession number NZ_ABFZ00000000.2, which is herein incorporated by reference in its entirety.

Nucleotide sequence information (including 16S rRNA sequence information) for microbial strain Bifidobacterium adolescentis, culture collection ATCC 15703, is available at DDBJ/EMBL/GENBANK accession number AP009256.1, which is herein incorporated by reference in its entirety.

Nucleotide sequence information (including 16S rRNA sequence information) for microbial strain Bifidobacterium longum subsp. infantis, culture collection ATCC 15697, is available at DDBJ/EMBL/GENBANK accession number CP001095.1, which is herein incorporated by reference in its entirety.

Nucleotide sequence information (including 16S rRNA sequence information) for microbial strain Bifidobacterium longum, culture collection ATCC 15697, is available at DDBJ/EMBL/GENBANK accession number CP001095.1, which is herein incorporated by reference in its entirety.

Nucleotide sequence information (including 16S rRNA sequence information) for microbial strain Blautia hydrogenotrophica, culture collection DSM 10507, is available at DDBJ/EMBL/GENBANK accession number NZ_ACBZ00000000.1, which is herein incorporated by reference in its entirety.

Nucleotide sequence information (including 16S rRNA sequence information) for microbial strain Blautia producta, culture collection ATCC 27340, is available at DDBJ/EMBL/GENBANK accession number ARETO1, which is herein incorporated by reference in its entirety.

Nucleotide sequence information (including 16S rRNA sequence information) for microbial strain Butyrivibrio fibrisolvens, culture collection ATCC 19171, is available at DDBJ/EMBL/GenBank accession number U41172.1, which is herein incorporated by reference in its entirety.

Nucleotide sequence information (including 16S rRNA sequence information) for microbial strain Butyrivibrio fibrisolvens, 16.4, is available at DDBJ/EMBL/GenBank accession number AJ250365.2, which is herein incorporated by reference in its entirety.

Nucleotide sequence information (including 16S rRNA sequence information) for microbial strain Butyrivibrio fibrisolvens, OB 156, is available at DDBJ/EMBL/GenBank accession number U41168.1, which is herein incorporated by reference in its entirety.

Nucleotide sequence information (including 16S rRNA sequence information) for microbial strain Butyrate-producing bacterium, A2-232, is available at DDBJ/EMBL/GenBank accession number AY305305.1, which is herein incorporated by reference in its entirety.

Nucleotide sequence information (including 16S rRNA sequence information) for microbial strain Butyrate-producing bacterium, SS3/4, is available at DDBJ/EMBL/GenBank accession number AY305316.1, which is herein incorporated by reference in its entirety.

Nucleotide sequence information (including 16S rRNA sequence information) for microbial strain Clostridium acetobutylicum, culture collection ATCC 824, is available at DDBJ/EMBL/GENBANK accession number AE001437.1, which is herein incorporated by reference in its entirety.

Nucleotide sequence information (including 16S rRNA sequence information) for microbial strain Clostridium acetobutylicum, culture collection DSM 792, is available at DDBJ/EMBL/GENBANK accession number X78070.1, which is herein incorporated by reference in its entirety.

Nucleotide sequence information (including 16S rRNA sequence information) for microbial strain Clostridium beijerinckii, culture collection NCIMB 8052, is available at DDBJ/EMBL/GENBANK accession number CP000721.1, which is herein incorporated by reference in its entirety.

Nucleotide sequence information (including 16S rRNA sequence information) for microbial strain Clostridium sporogenes, culture collection DSM 795, is available at DDBJ/EMBL/GENBANK accession number X68189.1, which is herein incorporated by reference in its entirety.

Nucleotide sequence information (including 16S rRNA sequence information) for microbial strain Clostridium tetani, is available at DDBJ/EMBL/GENBANK accession number X74770.1, which is herein incorporated by reference in its entirety.

Nucleotide sequence information (including 16S rRNA sequence information) for microbial strain Collinsella aerofaciens, culture collection ATCC 25986, is available at DDBJ/EMBL/GENBANK accession number AE001437.1, which is herein incorporated by reference in its entirety.

Nucleotide sequence information (including 16S rRNA sequence information) for microbial strain Coprococcus, butyrate-producing bacterium L2-50, is available at DDBJ/EMBL/GENBANK accession number AJ270491.2, which is herein incorporated by reference in its entirety.

Nucleotide sequence information (including 16S rRNA sequence information) for microbial strain Coprococcus comes, culture collection ATCC 27758, is available at DDBJ/EMBL/GENBANK accession number ABVRO1, which is herein incorporated by reference in its entirety.

Nucleotide sequence information (including 16S rRNA sequence information) for microbial strain Coprococcus eutactus, culture collection ATCC 27759, is available at DDBJ/EMBL/GENBANK accession number EF031543.1, which is herein incorporated by reference in its entirety.

Nucleotide sequence information (including 16S rRNA sequence information) for microbial strain Eubacterium cylindroides, butyrate-producing bacterium T2-87, is available at DDBJ/EMBL/GenBank accession number AY305306.1, which is herein incorporated by reference in its entirety.

Nucleotide sequence information (including 16S rRNA sequence information) for microbial strain Eubacterium cylindroides, butyrate-producing bacterium SM7/11, is available at DDBJ/EMBL/GenBank accession number AY305313.1, which is herein incorporated by reference in its entirety.

Nucleotide sequence information (including 16S rRNA sequence information) for microbial strain Eubacterium dolichum, culture collection DSM 3991, is available at DDBJ/EMBL/GenBank accession number L34682.2, which is herein incorporated by reference in its entirety.

Nucleotide sequence information (including 16S rRNA sequence information) for microbial strain Anerobutyricum hallii, butyrate-producing bacterium L2-7, is available atDDBJ/EMBL/GenBank accession number AJ270490.2, which is herein incorporated by reference in its entirety.

Nucleotide sequence information (including 16S rRNA sequence information) for microbial strain Anerobutyricum hallii, butyrate-producing bacterium SM6/1, is available at DDBJ/EMBL/GenBank accession number AY305318.1, which is herein incorporated by reference in its entirety.

Nucleotide sequence information (including 16S rRNA sequence information) for microbial strain Anerobutyricum hallii, culture collection ATCC 27751, is available at DDBJ/EMBL/GenBank accession number L34621.2, which is herein incorporated by reference in its entirety.

Nucleotide sequence information (including 16S rRNA sequence information) for microbial strain Eubacterium limosum, culture collection ATCC 5486, is available at DDBJ/EMBL/GenBank accession number NZ_CP019962.1, which is herein incorporated by reference in its entirety.

Nucleotide sequence information (including 16S rRNA sequence information) for microbial strain Eubacterium rectale, A1-86, is available at DDBJ/EMBL/GenBank accession number AJ270475.2, which is herein incorporated by reference in its entirety.

Nucleotide sequence information (including 16S rRNA sequence information) for microbial strain Eubacterium rectale, culture collection ATCC 33656, is available at DDBJ/EMBL/GENBANK accession number NC_012781.1, which is herein incorporated by reference in its entirety.

Nucleotide sequence information (including 16S rRNA sequence information) for microbial strain Eubacterium ventriosum, culture collection ATCC 27560, is available at DDBJ/EMBL/GenBank accession number L34421.2, which is herein incorporated by reference in its entirety.

Nucleotide sequence information (including 16S rRNA sequence information) for microbial strain Faecalibacterium prausnitzii, butyrate producing bacterium M21/2, is available at DDBJ/EMBL/GENBANK accession number AY305307.1, which is herein incorporated by reference in its entirety.

Nucleotide sequence information (including 16S rRNA sequence information) for microbial strain Faecalibacterium prausnitzii is available at DDBJ/EMBL/GENBANK accession number FP929046.1, which is herein incorporated by reference in its entirety.

Nucleotide sequence information (including 16S rRNA sequence information) for microbial strain Faecalibacterium prausnitzii is available at DDBJ/EMBL/GENBANK accession number GG697168.2, which is herein incorporated by reference in its entirety.

Nucleotide sequence information (including 16S rRNA sequence information) for microbial strain Fibrobacter succino genes subsp. succino genes is available at DDBJ/EMBL/GENBANK accession number CP002158.1, which is herein incorporated by reference in its entirety.

Nucleotide sequence information (including 16S rRNA sequence information) for microbial strain Clostridium butyricum, culture collection DSM 10702, is available at DDBJ/EMBL/GENBANK accession number NZ_AUJNO1000001.1, which is herein incorporated by reference in its entirety.

Nucleotide sequence information (including 16S rRNA sequence information) for microbial strain Clostridium indolis, culture collection DSM 755, is available at DDBJ/EMBL/GENBANK accession number NZ_AZUI01000001.1, which is herein incorporated by reference in its entirety.

Nucleotide sequence information (including 16S rRNA sequence information) for microbial strain Anerobutyricum hallii, culture collection DSM 3353, is available at DDBJ/EMBL/GENBANK accession number ACEP01000175.1, which is herein incorporated by reference in its entirety.

Nucleotide sequence information (including 16S rRNA sequence information) for microbial strain Roseburia faecis, M72/1, is available at DDBJ/EMBL/GenBank accession number AY305310.1, which is herein incorporated by reference in its entirety.

Nucleotide sequence information (including 16S rRNA sequence information) for microbial strain Roseburia hominis, type strain A2-183T, is available at DDBJ/EMBL/GenBank accession number AJ270482.2, which is herein incorporated by reference in its entirety.

Nucleotide sequence information (including 16S rRNA sequence information) for microbial strain Roseburia intestinalis, L1-82, is available at DDBJ/EMBL/GenBank accession number AJ312385.1, which is herein incorporated by reference in its entirety.

Nucleotide sequence information (including 16S rRNA sequence information) for microbial strain Roseburia inulinivorans, type strain A2-194T, is available at DDBJ/EMBL/GenBank accession number AJ270473.3, which is herein incorporated by reference in its entirety.

Nucleotide sequence information (including 16S rRNA sequence information) for microbial strain Roseburia inulinivorans, culture collection DSM 16841, is available at DDBJ/EMBL/GENBANK accession number NZ_ACFY01000179.1, which is herein incorporated by reference in its entirety.

Nucleotide sequence information (including 16S rRNA sequence information) for microbial strain Ruminococcus flavefaciens, culture collection ATCC 19208, is available at DDBJ/EMBL/GENBANK accession number K1912489.1, which is herein incorporated by reference in its entirety.

Nucleotide sequence information (including 16S rRNA sequence information) for microbial strain Ruminococcus gnavus, culture collection ATCC 29149, is available at DDBJ/EMBL/GENBANK accession number AAYG02000043.1, which is herein incorporated by reference in its entirety.

### BRIEF DESCRIPTION OF THE DRAWINGS

The patent application may contain at least one drawing executed in color. Copies of any color drawings will be provided upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings which consist of:

FIG. 1a-1e: Outline of study, analyses, and experiments. a. Clinical study overview including especially the ITT (n=76) and PP (n=58) participant totals, with T2D defined as fasting glucose≥126 mg/dL or HbA1c≥6.8%. b. The fasting plasma specimens were collected during the same visit as the originally-described glucose control endpoints. c. Of these 58 participants, 51 had successfully collected blood plasma pairs suitable for the indicated targeted and untargeted metabolomics analyses. d. Some of the observations derived from metabolomics were complemented with in vitro monoculture experiments using the formulation strains and additional metabolomics or growth dynamics measurements. e. Summary of probiotic strains included in the intervention formulations WBF-010 or WBF-011. Entries under each formulation identifier column indicate the approximate viable cell count per daily dose (CFU-equivalent), or absence if blank. Typical in vitro short-chain fatty acid production is indicated for each strain, with approximate ratios at late-log growth phase in VEG (AMUC) or PYG. Complete genome sequencing of each strain was performed, annotated, and deposited in GenBank with the indicated accession numbers.

FIG. 2a-2d: Summary of targeted measurements of short chain fatty acids in human plasma after fasting. a. Change in fasting circulating (plasma) concentrations [μM] for the three major short chain fatty acids (SCFAs). Line ranges summarize the 95% within-group confidence interval (Wilcoxon Rank Sum test), with nominal significance indicated above the respective group (star) or between-group comparison (bracket with star). Single and double stars indicate the one-sided significance of p<0.05 and p=0.007, respectively. b. Scatter plot of human plasma butyrate [μM] versus fecal butyrate [mM] corresponding to specimens collected at the same clinical visit. Line indicates the robust linear regression, with a green shading and p-value shown for the only slope coefficient to reach nominal significance, p=0.003. c. Scatter plot of the change in participant HbA1c versus their change in plasma butyrate [μM]. Due to the confounding variable of SFU drug use (see below), participants using SFU drugs are highlighted in gray and omitted from the robust linear regressions. In both (b) and (c) the gray ribbon denotes the 95% confidence region for the linear regression. d. Heatmap of Spearman's correlation between the participant-wise changes in metabolic measures (top seven rows) and major SCFAs (bottom three rows). Correlations were separately estimated for each study arm. Correlation magnitude and direction is indicated by the provided shading legend. ‘AUC_inc’ and ‘AUC_tot’ prefixes correspond to the incremental and total area under the curve of the oral meal tolerance test, respectively. For clarity, correlations with nominal p>0.4 are set to zero (white shading) irrespective of incidental correlation value.

FIG. 3a-3e: Plasma bile acids and evidence of direct conversion by formulation strains. a. Changes in selected bile acids from untargeted metabolomics, as log 2 ratio. Panel label indicates the group corresponding to the unconjugated form. Brackets highlight nominal statistical significance in between-group comparison (UDCA: p=0.016, G-UDCA: p=0.006), while (*) highlights within-group nominal significance (WBF-011, UDCA: p=0.089, G-UDCA: p=0.045). Shown in panels (a)-(c) are Placebo, WBF-010, and WBF-011 groups, respectively. b. Changes in selected bile acids in targeted data, in micromoles per liter. Ordered as in (a) for comparison. Brackets highlight nominal statistical significance in the between-group comparison (UDCA: p=0.075, G-UDCA: p=0.066). c. Plasma total bile acids. A light color highlights the beginning of the reference range for (intermediate) hyperbiluremia (10 μM). A gray horizontal line indicates the study grand median at baseline, while a short solid horizontal bar indicates the groupwise median at each timepoint. d. Summary of bile acids detected via untargeted metabolomics in a pilot in vitro monoculture survey. BINF, CBEI, CBUT and EHAL strains were grown in identical rich medium (PYG) amended with 50 μM each of human primary bile acids, cholic acid and CDCA. Color scale hew corresponds to negative or positive log 10-ratio values, a decrease or increase in concentration of the specimen relative to the uninoculated medium, respectively. e. Summary of UDCA synthesis during in vitro monoculture of formulation strains in media amended with 50 μM of the indicated primary bile acid. Only CBUT produced non-negligible UDCA. Shading emphasizes detected primary and secondary human bile acids, respectively. Change in concentration is calculated as the average concentration measured in the uninoculated medium subtracted from the volume-weighted concentration of the endpoint cell pellet and supernatant.

FIG. 4a-4e: Summary of untargeted metabolomics from fasting plasma. a. Distribution of the median participant-wise log 2-ratios of each metabolite. b. QQ-plot with 1-percentile steps for each probiotic intervention arm versus placebo. Shading as in (a). c Principal Component Analysis (PCA on the participant-wise log 2-ratios of each metabolite's untargeted value. Each point represents a participant, with shading by arm as in panel (A). Metabolites with low detection prevalence across the cohort were excluded (1156 of 1340 metabolites included). d. Volcano-plot summarizing the between-group multiple testing (Wilcoxon Rank Sum, two-sample, two-sided) of the participant-wise log 2-ratio of each metabolite. Vertical axis indicates the nominal p-value of each metabolite, while the horizontal axis indicates the estimated effect, structured as the Placebo group subtracted from the Formulation group (either WBF-011 or WBF-010). The full volcano scatterplot is repeated as light gray points in separate panels that have an additional layer of green- or blue-shaded points, with each panel emphasizing a different metabolite group of prior interest and apparent coordinated change. e. Summary of the within-group changes (participant-wise log 2-ratios) for each study arm by metabolite and metabolite group highlighted in (d). Diamond and linerange indicates the group median and Wilcoxon 95% confidence interval, respectively.

FIG. 5a-5c: Sulfonylurea drugs: use stratifies glucose control endpoints, and some formulation strains are inhibited by SFUs in a drug-specific pattern. a. Participant change in HbA1c versus study arm, with metabolomics-enhanced SFU drug usage status indicated by color shading. Gold and black color respectively indicate participants that are believed to have used, or not-used, SFU drug during the study as determined from the clinical record, direct observation of SFU in blood plasma, or both. Cross-bar indicates the value of each sub-group mean. b. Summary of the re-estimation of glucose control endpoint statistics in the comparison of WBF-011 group (the five-strain formulation) versus Placebo group (shown as WBF-011-Placebo). ‘Per protocol’ refers to the cohort that successfully completed the study, as described previously. ‘No SFU: Clinic’ refers to the results of between-group comparison among participants not known to use SFU according to the clinical record. This is equivalent to the confounding effect described in the original study. ‘No SFU: Clinic or Plasma’ is the same analysis, but with additional participants omitted where SFU was detected in metabolomics. c. Representative growth curves (OD 600 nm versus time in hours) for formulation strains with or without the presence of the indicated SFU drug in mVEG (AMUC), mPYG (CBEI, CBUT), or PYG (BINF, EHAL). Panel rows from top to bottom represent a 2-fold increasing concentration of the indicated SFU, with the millimolar concentration labeled in the top-left corner. Each black curve is a separate replicate inoculated at the same time as other curves for that strain, including positive controls. Green and blue regions indicate the range occupied by no-SFU positive controls in the same medium, with or without the final volumetric fraction of DMSO (3% for CBUT, 2% for all others), respectively. OD 600 nm values have been spline-smoothed and baseline-subtracted. To improve interpretability, each curve has been filtered to display just the period of continuous monotonic increase (growth).

### DETAILED DESCRIPTION

### Definitions

As used in the specification and claims, the singular forms “a”, “an” and “the” include plural references unless the context clearly dictates otherwise. For example, the term “a sample” includes a plurality of samples, including mixtures thereof.

The terms “microbes” and “microorganisms” are used interchangeably herein and can refer to bacteria, archaea, eukaryotes (e.g., protozoa, fungi, yeast), and viruses, including bacterial viruses (i.e., phage).

The term “microbiome”, “microbiota”, and “microbial habitat” are used interchangeably herein and can refer to the ecological community of microorganisms that live on or in a subject's body. The microbiome can be comprised of commensal, symbiotic, and/or pathogenic microorganisms. Microbiomes can exist on or in many, if not most parts of the subject. Some non-limiting examples of habitats of microbiome can include: body surfaces, body cavities, body fluids, the gut, the colon, skin surfaces and pores, vaginal cavity, umbilical regions, conjunctival regions, intestinal regions, the stomach, the nasal cavities and passages, the gastrointestinal tract, the urogenital tracts, saliva, mucus, and feces.

The term “pharmaceutical formulation” is any composition or formulation designed for administration to a subject. Such formulations may or may not meet the safety, efficacy, or other requirements for human use or approval by the FDA or other approval body or institution.

The term “prebiotic” as used herein can be a general term to refer to chemicals and/or ingredients that can affect the growth and/or activity of microorganisms in a host (e.g. can allow for specific changes in the composition and/or activity in the microbiome). Prebiotics can confer a health benefit on the host. Prebiotics can be selectively fermented, e.g. in the colon. Some non-limiting examples of prebiotics can include: complex carbohydrates, complex sugars, resistant dextrins, resistant starch, amino acids, peptides, nutritional compounds, biotin, polydextrose, fructooligosaccharide (FOS), galactooligosaccharides (GOS), inulin, lignin, psyllium, chitin, chitosan, gums (e.g. guar gum), high amylose cornstarch (HAS), cellulose, β-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), green banana, Reishi, tapioca, oats, pectin, potato or extracts thereof. Prebiotics can be found in foods (e.g. acacia gum, guar seeds, brown rice, rice bran, barley hulls, chicory root, Jerusalem artichoke, dandelion greens, garlic, leek, onion, asparagus, wheat bran, oat bran, baked beans, whole wheat flour, banana), and breast milk. Prebiotics can also be administered in other forms (e.g. capsule or dietary supplement).

The term “probiotic” as used herein can mean one or more microorganisms which, when administered appropriately, can confer a health benefit on the host or subject. Some non-limiting examples of probiotics include: Akkermansia muciniphila, Anaerostipes caccae, Bacteroides finegoldii, Bacteroides ovatus, Bacteroides stercoris, Eubactrium hallii, Bifidobacterium bifidum, Bifidobacterium infantis, Bifidobacterium longum, Blautia hydrogenotrophica, Blautia producta, Butyrivibrio fibrisolvens, Clostridium acetobutylicum, Clostridium aminophilum, Clostridium beijerinckii, Clostridium butyricum, Clostridium colinum, Clostridium indolis, Clostridium innocuum, Clostridium orbiscindens, Enterococcus faecium, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faeccis, Roseburia hominis, Roseburia intestinalis, Collinsella aerofaciens, Coprococcus comes, Eubacterium limosum, and Ruminococcus faecis, and all combinations thereof.

The terms “determining”, “measuring”, “evaluating”, “assessing,” “assaying,” and “analyzing” can be used interchangeably herein and can refer to any form of measurement, and include determining if an element is present or not. (e.g., detection). These terms can include both quantitative and/or qualitative determinations. Assessing may be relative or absolute. These terms can include use of the algorithms and databases described herein. “Detecting the presence of” can include determining the amount of something present, as well as determining whether it is present or absent. The term “genome assembly algorithm” as used herein, refers to any method capable of aligning sequencing reads with each other (de novo) or to a reference (re-sequencing) under conditions that a complete sequence of the genome may be determined.

The term “genome” as used herein, can refer to the entirety of an organism's hereditary information that is encoded in its primary DNA sequence. The genome includes both the genes and the non-coding sequences. For example, the genome may represent a microbial genome. The genetic content of the microbiome can comprise: genomic DNA, RNA, and ribosomal RNA, the epigenome, plasmids, and all other types of genetic information found in the microbes that comprise the microbiome.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand. The nucleic acid sequence can be made up of adenine, guanine, cytosine, thymine, and uracil (A, T, C, G, and U) as well as modified versions (e.g. N6-methyladenosine, 5-methylcytosine, etc.).

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence.

The term “sequencing” as used herein refers to sequencing methods for determining the order of the nucleotide bases—A, T, C, G, and U—in a nucleic acid molecule (e.g., a DNA or RNA nucleic acid molecule.

The term “biochip” or “array” can refer to a solid substrate having a generally planar surface to which an adsorbent is attached. A surface of the biochip can comprise a plurality of addressable locations, each of which location may have the adsorbent bound there. Biochips can be adapted to engage a probe interface, and therefore, function as probes. Protein biochips are adapted for the capture of polypeptides and can comprise of surfaces having chromatographic or biospecific adsorbents attached thereto at addressable locations. Microarray chips are generally used for DNA and RNA gene expression detection.

The term “barcode” as used herein, refers to any unique, non-naturally occurring, nucleic acid sequence that may be used to identify the originating genome of a nucleic acid fragment.

The terms “subject,” “individual,” “host,” and “patient” can be used interchangeably herein and refer to any animal subject, including: humans, mammals, laboratory animals, livestock, and household pets. The subject can host a variety of microorganisms. The subject can have different microbiomes in various habitats on and in their body. The subject may be diagnosed or suspected of being at high risk for a disease. The subject may have a microbiome state that is contributing to a disease (a dysbiosis). In some cases, the subject is not necessarily diagnosed or suspected of being at high risk for the disease. In some instances a subject may be suffering from an infection or at risk of developing or transmitting to others an infection.

The terms “treatment” or “treating” are used interchangeably herein. These terms can, but not necessarily, refer to an approach for obtaining beneficial or desired results including but not limited to a therapeutic benefit and/or a prophylactic benefit. A therapeutic benefit can mean eradication or amelioration of the underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The terms “16S”, “16S ribosomal subunit”, and “16S ribosomal RNA (rRNA)” can be used interchangeably herein and can refer to a component of a small subunit (e.g., 30S) of a prokaryotic (e.g., bacteria, archaea) ribosome. The 16S rRNA is highly conserved evolutionarily among species of microorganisms. Consequently, sequencing of the 16S ribosomal subunit can be used to identify and/or compare microorganisms present in a sample (e.g., a microbiome).

The term “spore” as used herein can refer to a viable cell produced by a microorganism to resist unfavorable conditions such as high temperatures, humidity, and chemical agents. A spore can have thick walls that allow the microorganism to survive harsh conditions for extended periods of time. Under suitable environmental conditions, a spore can germinate to produce a living form of the microorganism that is capable of reproduction and all of the physiological activities of the microorganism.

The term “homoacetogen” or acetogen refers to a microorganism that generates acetate (CH3COO—) as an end product of anaerobic respiration or fermentation. In some embodiments, the microorganisms are bacteria. These microbes perform anaerobic respiration and carbon fixation simultaneously through the reductive acetyl coenzyme A (acetyl-CoA) pathway (also known as the Wood-Ljungdahl pathway).

Compositions comprising microbes such as probiotics can confer a variety of beneficial effects on a subject. Examples of these beneficial effects can include reduction of pain, immunomodulatory features, regulation of cell proliferation, the ability to promote normal physiologic development of the mucosal epithelium, and enhancement of human nutrition. Microbial-based compositions can be administered as a therapeutic to a subject suffering from a microbiome-related health condition or disorder. Microbial-based compositions can be administered as a therapeutic to a subject so as to treat one or more disorders which are not related to the microbiome.

### Microbial Compositions and Formulations

Compositions or formulations of the disclosure can be administered as pharmaceutical formulations, therapeutic composition, dietary supplements, nutritional supplements, medical probiotics, or a medical food. In some cases, the composition is administered as a pharmaceutical formulation. In some cases, the composition is administered as a nutritional supplement. In some cases, the composition is administered as a dietary supplement. In some cases, the composition is administered as a medical food. In some cases, the composition is administered as a medical probiotic. In some cases, a composition (e.g., a dietary supplement, a nutritional supplement, a medical probiotic, or a medical food) can be administered orally, for example, as a capsule, pill, or tablet.

In embodiments, disclosed herein are formulations comprising one or more microbes selected from the group consisting of Akkermansia sp., Bifidobacterium sp., Clostridium sp., Anerobutyricum sp., and any combination thereof. As used herein, “sp.” stands for “species” and refers to all species of the recited genus that the term follows. Included are compositions comprising 1, 2, 3, or all 4 of the different genera recited. In further embodiments, such formulations can be used to treat or manage an inflammatory gut disorder, a liver disorder, a cholesterol metabolism disorder, a FXR-dependent cancer, a gallbladder disorder. In some embodiments, the formulation can be used to treat cancer. Examples of such inflammatory gut disorders include, but are not limited to, irritable bowel syndrome, inflammatory bowel disease, ulcerative colitis, diarrhea, constipation, leaky intestine, and/or Crohn's disease. Examples of liver disorders include but are not limited to alcohol-induced steatosis, non-alcoholic hepatic steatosis (NASH), steatohepatitis, non-alcoholic fatty liver disease (NAFLD), cirrhosis, and primary sclerosing cholangitis. Examples of cholesterol metabolism disorders include but are not limited to familial hypercholesterolemia, non-familial hypercholesterolemia, polygenic hypercholesterolemia, familial combined hyperlipidemia, excess small dense LDL. Examples of cancers include but are not limited to breast cancer, colorectal cancer, liver cancer, esophageal cancer, and pancreatic cancer. Examples of a gallbladder disorder include but are not limited to gallstones, biliary disorders that affect the bile duct, production of bile acids.

In embodiments, the formulation increases production of GLP-1 levels in the plasma of a subject treated with the formulation.

In embodiments, the formulation increases UDCA levels or glyco-UDCA levels in a subject treated with the formulation.

In embodiments, the formulation increases UDCA levels or glyco-UDCA levels in the plasma of a subject treated with the formulation.

In embodiments, the formulation increases UDCA levels or a salt thereof in the plasma of a subject to whom the formulation was administered.

In embodiments, disclosed herein are formulations comprising one or more microbes selected from the group consisting of Akkermansia muciniphila, Bifidobacterium infantis, Clostridium butyricum, Clostridium beijerinckii, and Anerobutyricum hallii, and any combination thereof. Included are compositions comprising 1, 2, 3, 4, 5, 6, 7, 8, 9, 10, 11, 12, 13, 14, 15, 16, 17, 18, 19 or all 20 of the different species recited and any combination thereof. In further embodiments, such formulations can be used to treat an inflammatory gut disorder, a liver disorder, a cholesterol metabolism disorder, a FXR-dependent cancer, a gallbladder disorder. In some embodiments, the formulation can be used to treat cancer. Examples of such inflammatory gut disorders include, but are not limited to, irritable bowel syndrome, inflammatory bowel disease, ulcerative colitis, diarrhea, constipation, leaky intestine, and/or Crohn's disease. Examples of liver disorders include but are not limited to alcohol-induced steatosis, non-alcoholic hepatic steatosis (NASH), steatohepatitis, non-alcoholic fatty liver disease (NAFLD), cirrhosis, and primary sclerosing cholangitis. Examples of cholesterol metabolism disorders include but are not limited to familial hypercholesterolemia, non-familial hypercholesterolemia, polygenic hypercholesterolemia, familial combined hyperlipidemia, excess small dense LDL. Examples of cancers include but are not limited to breast cancer, colorectal cancer, liver cancer, esophageal cancer, and pancreatic cancer. Examples of a gallbladder disorder include but are not limited to gallstones, biliary disorders that affect the bile duct, production of bile acids.

In embodiments, the formulation increases production of GLP-1 levels in the plasma of a subject treated with the formulation.

In embodiments, the formulation increases UDCA levels or glyco-UDCA levels in the plasma of a subject treated with the formulation.

In embodiments, the formulation increases UDCA levels or a salt thereof in the plasma of a subject to whom the formulation was administered.

In embodiments, the formulation increases UDCA levels, glyco-UDCA levels, other salts of UDCA, or a combination thereof, in a subject treated with the formulation.

In some embodiments, the formulation increases medium chain fatty acids and carnitinylates thereof in a subject treated with the formulation.

In some further embodiments, the formulation decreases bilirubin degradation byproducts.

In embodiments, disclosed herein are formulations comprising one or more microbes having a 16S rRNA sequence comprising at least 95% identity to the full length of a 16S rRNA sequence of a microbe selected from the group consisting of Akkermansia muciniphila ATCC BAA-835, Bifidobacterium infantis ATCC 15697, Clostridium butyricum DSM 10702, Clostridium beijerinckii NCIMB 8052, and anerobutyricum hallii DSM 3353, and any combination thereof. Included are compositions comprising 1, 2, 3, 4, 5, 6, 7, 8, 9, 10, 11, 12, 13, 14, 15, 16, 17, 18, 19 or all 20 different microbes such that 1, 2, 3, 4, 5, 6, 7, 8, 9, 10, 11, 12, 13, 14, 15, 16, 17, 18, 19 or all 20 of the different 16S rRNA sequences are present in the composition. Certain embodiments include formulations, wherein the at least about 95% sequence identity is selected from the group consisting of: at least about 96%, at least about 97%, at least about 98%, at least about 99%, at least about 99.5%, and at least about 99.5% sequence identity to a rRNA sequence. In further embodiments, such formulations can be used to treat an inflammatory gut disorder, a liver disorder, a cholesterol metabolism disorder, a FXR-dependent cancer, a gallbladder disorder. Examples of liver disorders include but are not limited to alcohol-induced steatosis, non-alcoholic hepatic steatosis (NASH), steatohepatitis, non-alcoholic fatty liver disease (NAFLD), cirrhosis, and primary sclerosing cholangitis. Examples of cholesterol metabolism disorders include but are not limited to familial hypercholesterolemia, non-familial hypercholesterolemia, polygenic hypercholesterolemia, familial combined hyperlipidemia, excess small dense LDL. Examples of cancers include but are not limited to breast cancer, colorectal cancer, liver cancer, esophageal cancer, and pancreatic cancer. Examples of a gallbladder disorder include but are not limited to gallstones, biliary disorders that affect the bile duct, production of bile acids.

In embodiments, the formulation increases production of GLP-1 levels in the plasma of a subject treated with the formulation.

In embodiments, the formulation increases UDCA levels or glyco-UDCA levels in the plasma of a subject treated with the formulation.

In embodiments, the formulation increases UDCA levels or a salt thereof in the plasma of a subject to whom the formulation was administered.

In embodiments, the formulation increases UDCA levels, glyco-UDCA levels, other salts of UDCA, or a combination thereof, in a subject treated with the formulation.

In some embodiments, the formulation increases medium chain fatty acids and carnitinylates thereof in a subject treated with the formulation.

In some further embodiments, the formulation decreases bilirubin degradation byproducts.

In one embodiment, a composition to treat an inflammatory gut disorder, a liver disorder, a cholesterol metabolism disorder, a FXR-dependent cancer, a non-FXR dependent cancer, a gallbladder disorder comprises an isolated and/or purified microorganism population consisting of microbes with at least about: 95%, 96%, 97%, 98%, 99%, 99.5%, or 100% sequence identity to the 16SrRNA of Akkermansia muciniphila ATCC BAA-835.

In one embodiment, a composition to treat an inflammatory gut disorder, a liver disorder, a cholesterol metabolism disorder, a FXR-dependent cancer, a non-FXR dependent cancer, a gallbladder disorder comprises an isolated and/or purified microorganism population consisting of microbes with at least about: 95%, 96%, 97%, 98%, 99%, 99.5%, or 100% sequence identity to the 16SrRNA of Bifidobacterium infantis ATCC 15697.

In one embodiment, a composition to treat an inflammatory gut disorder, a liver disorder, a cholesterol metabolism disorder, a FXR-dependent cancer, a non-FXR dependent cancer, a gallbladder disorder comprises an isolated and/or purified microorganism population consisting of microbes with at least about: 95%, 96%, 97%, 98%, 99%, 99.5%, or 100% sequence identity to the 16SrRNA of Clostridium butyricum DSM 10702.

In one embodiment, a composition to treat an inflammatory gut disorder, a liver disorder, a cholesterol metabolism disorder, a FXR-dependent cancer, a non-FXR dependent cancer, a gallbladder disorder comprises an isolated and/or purified microorganism population consisting of microbes with at least about: 95%, 96%, 97%, 98%, 99%, 99.5%, or 100% sequence identity to the 16SrRNA of Clostridium beijerinckii NCIMB 8052.

In one embodiment, a composition to treat an inflammatory gut disorder, a liver disorder, a cholesterol metabolism disorder, a FXR-dependent cancer, a non-FXR dependent cancer, a gallbladder disorder comprises an isolated and/or purified microorganism population consisting of microbes with at least about: 95%, 96%, 97%, 98%, 99%, 99.5%, or 100% sequence identity to the 16SrRNA of anerobutyricum hallii DSM 3353.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Akkermansia muciniphila.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Bifidobacterium infantis.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Clostridium beijerinckii.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Clostridium butyricum.

In one embodiment, a composition comprises an isolated and/or purified microorganism population consisting of microbes with at least about: 70%, 75%, 80%, 85%, 87%, 90%, 91%, 92%, 93%, 94%, 95%, 96%, 97%, 98%, 99%, 99.5%, or 100% sequence identity to the 16SrRNA of Anerobutyricum hallii.

In one embodiment, a composition comprises microbes from 2 or more, 3 or more, four or more, or all five of the group consisting of Akkermansia muciniphila, Bifidobacterium infantis, Clostridium beijerinckii, Clostridium butyricum, and Anerobutyricum hallii.

In one embodiment, a composition comprises 2 or more, 3 or more, four or more, or all five different microbes of the group consisting of microbes having 16S rRNA sequence comprising at least 97% identity to the full length of a 16S rRNA sequence of Akkermansia muciniphila, Bifidobacterium infantis, Clostridium beijerinckii, Clostridium butyricum, and Anerobutyricum hallii.

In one embodiment, a composition comprises microbes from 2 or more, 3 or more, four or more, or all five of the group consisting of Akkermansia muciniphila ATCC BAA-835, Bifidobacterium infantis ATCC 15697, Clostridium butyricum DSM 10702, Clostridium beijerinckii NCIMB 8052, and Anerobutyricum hallii DSM 3353.

In one embodiment, a composition comprises Akkermansia muciniphila, Bifidobacterium infantis, Clostridium beijerinckii, Clostridium butyricum, and Anerobutyricum hallii.

In one embodiment, a composition comprises 16S rRNA sequence comprising at least 97% identity to the full length of a 16S rRNA sequence of Akkermansia muciniphila, Bifidobacterium infantis, Clostridium beijerinckii, Clostridium butyricum, and Anerobutyricum hallii.

In one embodiment, a composition comprises Akkermansia muciniphila ATCC BAA-835, Bifidobacterium infantis ATCC 15697, Clostridium butyricum DSM 10702, Clostridium beijerinckii NCIMB 8052, and Anerobutyricum hallii DSM 3353.

In one embodiment, a composition comprises Clostridium butyricum, and Akkermansia muciniphila.

In one embodiment, a composition comprises a 16S rRNA sequence comprising at least 97% identity to the full length of a 16S rRNA sequence of Clostridium butyricum, and Akkermansia muciniphila.

In one embodiment, a composition comprises Akkermansia muciniphila ATCC BAA-835, and Clostridium butyricum DSM 10702.

In one embodiment, a composition comprises Clostridium butyricum, anerobutyricum hallii, and Akkermansia muciniphila.

In one embodiment, a composition comprises a 16S rRNA sequence comprising at least 97% identity to the full length of a 16S rRNA sequence of Clostridium butyricum, anerobutyricum hallii, and Akkermansia muciniphila.

In one embodiment, a composition comprises Akkermansia muciniphila ATCC BAA-835, Anerobutyricum hallii DSM 3353, and Clostridium butyricum DSM 10702.

In one embodiment, a composition comprises Clostridium butyricum; Clostridium butyricum and Akkermansia muciniphila; Clostridium butyricum and Anerobutyricum hallii; Clostridium butyricum and Clostridium beijerinckii; Clostridium butyricum and Bifidobacterium infantis; Akkermansia muciniphila; Akkermansia muciniphila and Anerobutyricum hallii; Akkermansia muciniphila and Clostridium beijerinckii; Akkermansia muciniphila and Bifidobacterium infantis; Anerobutyricum hallii; Anerobutyricum hallii and Clostridium beijerinckii; Anerobutyricum hallii and Bifidobacterium infantis; Clostridium beijerinckii; Clostridium beijerinckii and Bifidobacterium infantis; or only Bifidobacterium infantis.

In one embodiment, a composition comprises Clostridium butyricum, Akkermansia muciniphila, and Anerobutyricum hallii; Clostridium butyricum, Akkermansia muciniphila and Clostridium beijerinckii; or Clostridium butyricum, Akkermansia muciniphila and Bifidobacterium infantis.

In one embodiment, a composition comprises Clostridium butyricum, Anerobutyricum hallii, Clostridium beijerinckii; Clostridium butyricum, Anerobutyricum hallii, Bifidobacterium infantis; or Clostridium butyricum, Clostridium beijerinckii, and Bifidobacterium infantis.

In one embodiment, a composition comprises Akkermansia muciniphila, Anerobutyricum hallii, and Clostridium beijerinckii; Akkermansia muciniphila, Anerobutyricum hallii, and Bifidobacterium infantis; or Akkermansia muciniphila, Clostridium beijerinckii, and Bifidobacterium infantis.

In one embodiment, a composition comprises Anerobutyricum hallii, Bifidobacterium infantis, and Clostridium beijerinckii.

In one embodiment, a composition comprises Clostridium butyricum, Akkermansia muciniphila, Anerobutyricum hallii, Clostridium beijerinckii; Clostridium butyricum, Akkermansia muciniphila, Anerobutyricum hallii, and Bifidobacterium infantis; Clostridium butyricum, Akkermansia muciniphila, Clostridium beijerinckii and Bifidobacterium infantis; Clostridium butyricum, Anerobutyricum hallii, Clostridium beijerinckii, and Bifidobacterium infantis; Akkermansia muciniphila, Anerobutyricum hallii, Clostridium beijerinckii, and Bifidobacterium infantis; or Clostridium butyricum, Akkermansia muciniphila, Anerobutyricum hallii, Clostridium beijerinckii, and Bifidobacterium infantis.

In one embodiment, a composition comprises Clostridium butyricum DSM 10702.

In one embodiment, a composition comprises a bacteria belonging to the genus Clostridia.

In one embodiment, a composition comprises a bacteria that expresses one or more enzymes having any one or both of 7α- and/or 7β-hydroxysteroid dehydrogenase (7αβ-HSDH) activity (ECC 1.1.1.159 and 1.1.1.201). In particular embodiments, the bacteria expressing any one or both of the enzymes is genetically modified. In other embodiments, such bacteria belong to the genus Clostridia. In further embodiments, the bacteria comprise a 16S rRNA sequence comprising at least 95% identity to the full length of a 16S rRNA sequence of Clostridium butyricum. In further embodiments, the bacteria comprise a 16S rRNA sequence comprising at least 97% identity to the full length of a 16S rRNA sequence of Clostridium butyricum.

In one embodiment, a composition comprises Akkermansia muciniphila ATCC BAA-835. In one embodiment, a composition comprises one or more of the microbes chosen from the group consisting of Akkermansia muciniphila ATCC BAA-835, Anerobutyricum hallii DSM 3353, and Clostridium butyricum DSM 10702.

Certain embodiments include a composition of any of the preceding embodiments, wherein the formulation further comprises one or more additional microbe strains having a 16S rRNA sequence comprising at least 95% identity to the full length of a 16S rRNA sequence of a microbe selected from the group consisting of Anaerostipes caccae, Bacteroides finegoldii, Bacteroides ovatus, Bacteroides stercoris, Anerobutyricum hallii, Bifidobacterium bifidum, Bifidobacterium infantis, Bifidobacterium longum, Blautia hydrogenotrophica, Blautia producta, Butyrivibrio fibrisolvens, Clostridium acetobutylicum, Clostridium aminophilum, Clostridium beijerinckii, Clostridium colinum, Clostridium indolis, Clostridium innocuum, Clostridium orbiscindens, Enterococcus faecium, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faeccis, Roseburia hominis, Roseburia intestinalis, Collinsella aerofaciens, Coprococcus comes, Eubacterium limosum, and Ruminococcus faecis, and all combinations thereof. Certain embodiments include formulations, wherein the at least about 95% sequence identity is selected from the group consisting of: at least about 96%, at least about 97%, at least about 98%, at least about 99%, at least about 99.5%, and at least about 99.5% sequence identity to a rRNA sequence.

A composition may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, or at least 50, or at least 75, or at least 100 types of microbes. A composition may comprise at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 45, or at most 50, or at most 75, or at most 100 types of microbes.

Provided herein are compositions that may be administered as pharmaceuticals, therapeutics, dietary or nutritional supplements, and/or cosmetics. One or more microorganisms described herein can be used to create a composition comprising an effective amount of the composition for treating a subject. The microorganisms can be in any formulation known in the art. Some non-limiting examples can include topical, capsule, pill, enema, liquid, injection, and the like. In some embodiments, the one or more strains disclosed herein may be included in a food or beverage product, cosmetic, or nutritional supplement.

In some embodiments, a composition as described herein comprises an enteric coating. The composition may be formulated as an enteric-coated pill. An enteric-coating can protect the contents of a formulation, for example, pill or capsule, from the acidity of the stomach and provide delivery to the ileum and/or upper colon regions. Non-limiting examples of enteric coatings include pH sensitive polymers (e.g., eudragit FS30D), methyl acrylate-methacrylic acid copolymers, cellulose acetate succinate, hydroxypropyl methyl cellulose phthalate, hydroxypropyl methylcellulose acetate succinate (e.g., hypromellose acetate succinate), polyvinyl acetate phthalate (PVAP), methyl methacrylate-methacrylic acid copolymers, shellac, cellulose acetate trimellitate, sodium alginate, zein, other polymers, fatty acids, waxes, shellac, plastics, and plant fibers.

The enteric coating can be designed to dissolve at any suitable pH. In some embodiments, the enteric coating is designed to dissolve at a pH greater than about pH 6.5 to about pH 7.0. In some embodiments, the enteric coating is designed to dissolve at a pH greater than about pH 6.5. In some embodiments, the enteric coating is designed to dissolve at a pH greater than about pH 7.0. The enteric coating can be designed to dissolve at a pH greater than about: 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, or 7.5 pH units.

A composition can be substantially free of preservatives. In some applications, the composition may contain at least one preservative. In particular embodiments, formulations as described herein may contain an effective amount of a preservative. An “effective” amount is any amount that preserves or increases the shelf life of the composition beyond what would be obtained if the preservative were not present in the formulation. Examples of such preservatives include, but are not limited to, Vitamin E, Vitamin C, butylatedhydroxyanisole (BHA). butylatedhydroxytoluene (BHT), disodium ethylenediaminetetraacetic acid (EDTA), polyphosphates, citric acid, benzoates, sodium benzoate, sorbates, propionets, and nitrites.

The formulation can include one or more active ingredients. Active ingredients include, but are not limited to, antibiotics, prebiotics, probiotics, glycans (e.g., as decoys that would limit specific bacterial/viral binding to the intestinal wall), bacteriophages, microorganisms, bacteria, and the like.

In some embodiments, the formulation comprises a prebiotic. In some embodiments, the prebiotic is inulin, green banana, Reishi, tapioca, oats, pectin, potato or extracts thereof, complex carbohydrates, complex sugars, resistant dextrins, resistant starch, amino acids, peptides, nutritional compounds, biotin, polydextrose, fructooligosaccharide (FOS), galactooligosaccharides (GOS), starch, lignin, psyllium, chitin, chitosan, gums (e.g. guar gum), high amylose cornstarch (HAS), cellulose, β-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), and any combination thereof. The prebiotic can serve as an energy source for the microbial formulation.

A formulation can be formulated for administration by a suitable method for delivery to any part of the gastrointestinal tract of a subject including oral cavity, mouth, esophagus, stomach, duodenum, small intestine regions including duodenum, jejunum, ileum, and large intestine regions including cecum, colon, rectum, and anal canal. In some embodiments, the composition is formulated for delivery to the ileum and/or colon regions of the gastrointestinal tract.

Pharmaceutical formulations can be formulated as a dietary supplement. Pharmaceutical formulations can be incorporated with vitamin supplements. pharmaceutical formulations can be formulated in a chewable form such as a probiotic gummy. Pharmaceutical formulations can be incorporated into a form of food and/or drink. Non-limiting examples of food and drinks where the microbial compositions can be incorporated include, for example, bars, shakes, juices, infant formula, beverages, frozen food products, fermented food products, and cultured dairy products such as yogurt, yogurt drink, cheese, acidophilus drinks, and kefir.

A formulation of the disclosure can be administered as part of a fecal transplant process. A formulation can be administered to a subject by a tube, for example, nasogastric tube, nasojejunal tube, nasoduodenal tube, oral gastric tube, oral jejunal tube, or oral duodenal tube. A formulation can be administered to a subject by colonoscopy, endoscopy, sigmoidoscopy, and/or enema.

In some embodiments, the composition is formulated such that the one or more microbes can replicate once they are delivered to the target habitat (e.g. the gut). In one non-limiting example, the microbial composition is formulated in a capsule or a pill, such that the capsule or pill has a shelf life of at least 1, 2, 3, 4, 5, 6, 7, 8, 9, 10, 11, or 12 months. In another non-limiting example, the storage of the microbial composition is formulated so that the microbes can reproduce once they are in the gut. In some embodiments, other components may be added to aid in the shelf life of the microbial composition. In some embodiments, one or more microbes may be formulated in a manner that is able to survive in a non-natural environment. For example, a microbe that is native to the gut may not survive in an oxygen-rich environment. To overcome this limitation, the microbe may be formulated in a pill that can reduce or eliminate the exposure to oxygen. Other strategies to enhance the shelf-life of microbes may include other microbes (e.g. if the composition comprises elements whereby one or more strains is helpful for the survival of one or more strains).

In some embodiments, one or more of the microbes are lyophilized (e.g., freeze-dried) and formulated as a powder, tablet, enteric-coated capsule (e.g. for delivery to ileum/colon), or pill that can be administered to a subject by any suitable route. The lyophilized formulation can be mixed with a saline or other solution prior to administration.

In some embodiments, a composition is formulated for oral administration, for example, as an enteric-coated capsule or pill, for delivery of the contents of the formulation to the ileum and/or colon regions of a subject.

In some embodiments, the composition is formulated for oral administration. In some embodiments, the composition is formulated as an enteric-coated pill or capsule for oral administration. In some embodiments, the composition is formulated for delivery of the microbes to the ileum region of a subject. In some embodiments, the composition is formulated for delivery of the microbes to the colon region (e.g. upper colon) of a subject. In some embodiments, the composition is formulated for delivery of the microbes to the ileum and colon regions of a subject.

In some embodiments, the composition is formulated as a population of spores. Spore-containing formulations can be administered by any suitable route described herein. Orally administered spore-containing formulations can survive the low pH environment of the stomach. The amount of spores employed can be, for example, from about 1% w/w to about 99% w/w of the entire formulation.

Formulations provided herein can include the addition of one or more agents to the therapeutics or cosmetics in order to enhance stability and/or survival of microbes in the formulation. Non-limiting example of stabilizing agents include genetic elements, glycerin, ascorbic acid, skim milk, lactose, tween, alginate, xanthan gum, carrageenan gum, mannitol, palm oil, and poly-L-lysine (POPL).

In some embodiments, a formulation comprises one or more recombinant microbes or microbes that have been genetically modified. In other embodiments, one or more microbes are not modified or recombinant. In some embodiments, the formulation comprises microbes that can be regulated, for example, a microbe comprising an operon or promoter to control microbial growth. Microbes as described herein can be produced, grown, or modified using any suitable methods, including recombinant methods.

A formulation can be customized for a subject. A custom formulation can comprise, for example, a prebiotic, a probiotic, an antibiotic, or a combination of active agents described herein. Data specific to the subject comprising for example age, gender, and weight can be combined with an analysis result to provide a therapeutic agent customized to the subject. For example, a subject's microbiome found to be low in a specific microbe relative to a sub-population of healthy subjects matched for age and gender can be provided with a therapeutic and/or cosmetic formulation comprising the specific microbe to match that of the sub-population of healthy subjects having the same age and gender as the subject.

Formulations provided herein can include those suitable for oral including buccal and sub-lingual, intranasal, topical, transdermal, transdermal patch, pulmonary, vaginal, rectal, suppository, mucosal, systemic, or parenteral including intramuscular, intraarterial, intrathecal, intradermal, intraperitoneal, subcutaneous, and intravenous administration or in a form suitable for administration by aerosolization, inhalation or insufflation.

A formulation can include carriers and/or excipients (including but not limited to buffers, carbohydrates, lipids, mannitol, proteins, polypeptides or amino acids such as glycine, antioxidants, bacteriostats, chelating agents, suspending agents, thickening agents and/or preservatives), metals (e.g., iron, calcium), salts, vitamins, minerals, water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, adjuvants, or binders, other pharmaceutically acceptable auxiliary substances as required to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, antioxidants, gums, coating agents, coloring agents, flavoring agents, dispersion enhancer, disintegrant, coating agents, plasticizers, preservatives, suspending agents, emulsifying agents, plant cellulosic material and spheronization agents, and any combination thereof.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

A pharmaceutical, therapeutic, nutritional, dietary, or cosmetic composition can be encapsulated within a suitable vehicle, for example, a liposome, a microspheres, or a microparticle. Microspheres formed of polymers or proteins can be tailored for passage through the gastrointestinal tract directly into the blood stream. Alternatively, the compound can be incorporated and the microspheres, or composite of microspheres, and implanted for slow release over a period of time ranging from days to months.

A pharmaceutical, therapeutic, or cosmetic composition can be formulated as a sterile solution or suspension. The compositions can be sterilized by conventional techniques or may be sterile filtered. The resulting aqueous solutions may be packaged for use as is, or lyophilized. The lyophilized preparation of the microbial composition can be packaged in a suitable form for oral administration, for example, capsule or pill.

The compositions can be administered topically and can be formulated into a variety of topically administrable compositions, such as solutions, suspensions, lotions, gels, pastes, medicated sticks, balms, creams, and ointments. Such compositions can contain solubilizers, stabilizers, tonicity enhancing agents, buffers and preservatives.

The compositions can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as a mixture of fatty acid glycerides, optionally in combination with cocoa butter, can be used.

Compositions can be formulated using one or more physiologically-acceptable carriers comprising excipients and auxiliaries, which facilitate processing of the microorganisms into preparations that can be used pharmaceutically. Formulation may be modified depending upon the route of administration chosen. Compositions described herein may be manufactured, for example, by means of conventional mixing, dissolving, granulating, vitrification, spray-drying, lyophilizing, dragee-making, levigating, encapsulating, entrapping, emulsifying or compression processes.

In some embodiments, the composition is manufactured in a dry form, for example, by spray-drying or lyophilization. In some embodiments, the formulation is prepared as a liquid capsule to maintain the liquid form of the microbes.

Compositions provided herein can be stored at any suitable temperature. The formulation can be stored in cold storage, for example, at a temperature of about −80° C., about −20° C., about −4° C., or about 4° C. The storage temperature can be, for example, about 0° C., about 1° C., about 2° C., about 3° C., about 4° C., about 5° C., about 6° C., about 7° C., about 8° C., about 9° C., about 10° C., about 12° C., about 14° C., about 16° C., about 20° C., about 22° C., or about 25° C. In some embodiments, the storage temperature is between about 2° C. to about 8° C. Storage of microbial compositions at low temperatures, for example from about 2° C. to about 8° C., can keep the microbes alive and increase the efficiency of the composition, for example, when present in a liquid or gel formulation. Storage at freezing temperature, below 0° C., with a cryoprotectant can further extend stability.

The pH of the composition can range from about 3 to about 12. The pH of the composition can be, for example, from about 3 to about 4, from about 4 to about 5, from about 5 to about 6, from about 6 to about 7, from about 7 to about 8, from about 8 to about 9, from about 9 to about 10, from about 10 to about 11, or from about 11 to about 12 pH units. The pH of the composition can be, for example, about 3, about 4, about 5, about 6, about 7, about 8, about 9, about 10, about 11, or about 12 pH units. The pH of the composition can be, for example, at least 3, at least 4, at least 5, at least 6, at least 7, at least 8, at least 9, at least 10, at least 11 or at least 12 pH units. The pH of the composition can be, for example, at most 3, at most 4, at most 5, at most 6, at most 7, at most 8, at most 9, at most 10, at most 11, or at most 12 pH units. If the pH is outside the range desired by the formulator, the pH can be adjusted by using sufficient pharmaceutically-acceptable acids and bases. In some embodiments, the pH of the composition is between about 4 and about 6.

Compositions containing microbes described herein can be administered for prophylactic and/or therapeutic treatments. In therapeutic applications, the compositions can be administered to a subject already suffering from a disease or condition, in an amount sufficient to cure or at least partially arrest the symptoms of the disease or condition, or to cure, heal, improve, or ameliorate the condition. Microbial compositions can also be administered to lessen a likelihood of developing, contracting, or worsening a condition. Amounts effective for this use can vary based on the severity and course of the disease or condition, previous therapy, the subject's health status, weight, and response to the drugs, and the judgment of the treating physician. An effective amount can be administered in one or more administrations.

In some embodiments, combining one or more microbes in a composition can provide a synergistic effect when administered to the individual. For example, administration of a first microbe may be beneficial to a subject and administration of a second microbe may be beneficial to a subject but when the two microbes are administered together to a subject, the benefit is greater than the either benefit alone.

Different types of microbes in a composition can be present in the same amount or in different amounts. For example, the ratio of two microbes in a composition can be about 1:1, 1:2, 1:5, 1:10, 1:25, 1:50, 1:100, 1:1000, 1:10,000, or 1:100,000.

In some embodiments, a composition comprises at least one primary fermenter (e.g., a microbe that generates a substrate such as lactate or acetate) and at least one secondary fermenter (e.g., a microbe that utilizes the substrate produced by the primary fermenter to produce a secondary product such as butyrate). In some embodiments, a composition comprises at least one primary fermenter, at least one secondary fermenter, and at least one prebiotic (e.g., to serve as an energy source for the primary and/or secondary fermenter).

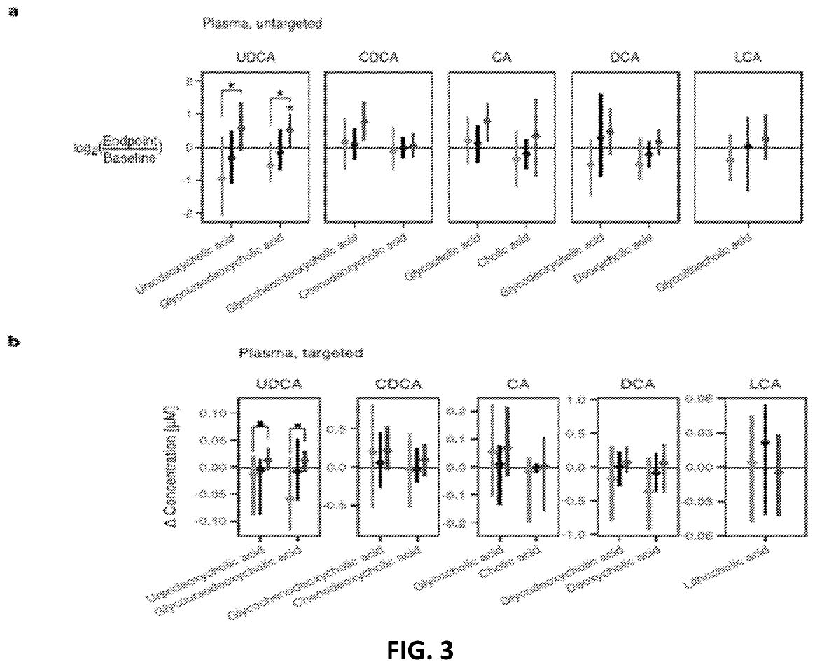

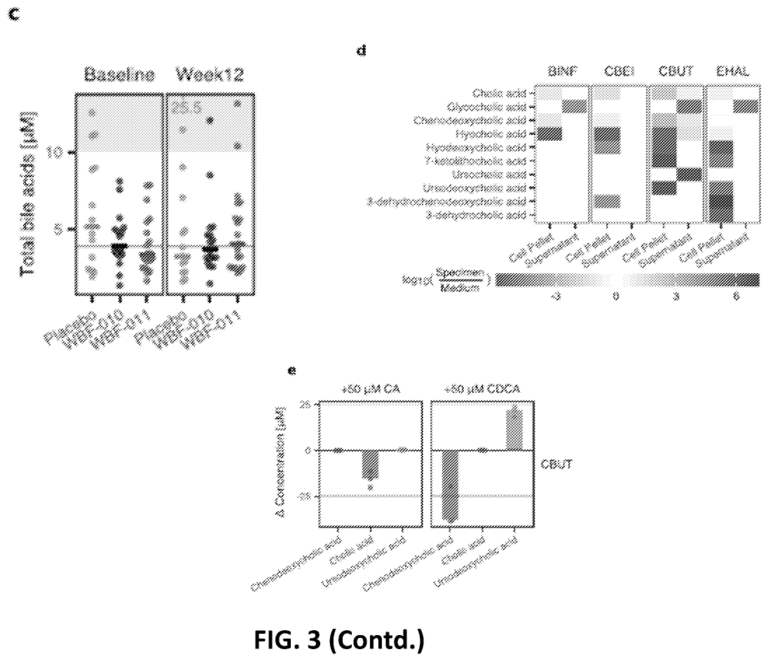

In some embodiments, a composition comprises at least one microbe that produces butyrate and produces at least one enzyme that converts at least one primary bile acid to at least one secondary bile acid. Examples of such secondary bile acids include, but are not limited to, UDCA or tauro-UDCA, glyco-UDCA or others as shown in FIG. 3b. In some embodiments, the produced butyrate and UDCA, synergistically, stimulates secretion of GLP-1 in L-cells of intestine.

In some embodiments, a composition comprises at least one microbe that produces butyrate and at least a second microbe that produces at least one enzyme that converts at least one primary bile acid to at least one secondary bile acid. Examples of such secondary bile acids include, but are not limited to, UDCA or tauro-UDCA, glyco-UDCA or others as shown in FIG. 3b. In some embodiments, the produced butyrate and UDCA, synergistically, antagonizes FXR in intestinal L cells.

Microbes may be produced in any suitable medium for growth, some non-limiting examples include: RCM, GYT veg, BHI, PYGveg, nutrient media, minimal media, selective media, differential media, and transport media. The growth medium can comprise a trace mineral. The growth medium can comprise a salt. The growth medium can comprise a vitamin. The growth medium can comprise a buffer. The pH of a growth medium can be, for example, about 7. The pH of a growth medium can be, for example, about 3, about, 4, about, 5, about 6, about 7, or about 8. The growth medium can improve the maximum density a microbial strain can grow to. The growth medium can allow for higher strain concentrations. The growth medium can buffer acid production by a microbial strain, which can minimize the inhibitory effect of, for example, very low pH.

In some embodiments, the media used for microbial culture is a vegetable-based media that is free of any animal or dairy based ingredients or derivatives. In another embodiment, the media is a meat-free media that is free of any animal-derived components. In an embodiment, the media is a culture medium having a pH of at least 6 and at most 8. In an embodiment, culture medium comprises one or more of a sugar, a yeast extract, a plant-derived peptone, plant-derived hydrolysate, cysteine, magnesium, calcium, potassium, and a vitamin; and lacks any animal, meat, or dairy based ingredients or derivatives. In an embodiment, the microbes are cultured under anaerobic conditions. In an embodiment, the microbes are lyophilized under anaerobic conditions.

### Methods of Treating a Subject

The disclosure provides methods for treating a subject or managing a health condition. Altering the composition of a microbiome in a subject can have desired health consequences. Compositions of the disclosure can be administered as a therapeutic, nutritional/dietary supplement, and/or a cosmetic for treating a health condition. Treatments designed to alter the host microbiome(s) can result in a reduction of patient symptoms, prevention of disease, and or treatment of the disease or health condition.

Compositions disclosed herein can be used for the dietary management of an inflammatory gut disorder. Compositions disclosed herein can be used for the dietary management of irritable bowel syndrome, inflammatory bowel disease, ulcerative colitis, diarrhea, constipation, leaky intestine, and/or Crohn's disease.

Compositions disclosed herein can be used for the dietary management of pain in response to liver distension in a subject.

Compositions disclosed herein can be used for dietary management of a liver disorder, a cholesterol metabolism disorder, obesity, dyslipidemia, FXR receptor dependent cancer, a non-FXR dependent cancer. The contents of Huang et al. (Acta Pharmacologica Sinica (2015) 36:37-43 (FXR and liver carcinogenesis)) is herein incorporated by reference in its entirety.

Compositions disclosed herein can be used for dietary management of gallbladder malfunction. The malfunction may be caused by gallstones or biliary duct issues.

Compositions disclosed herein increases anti-inflammatory response in a subject who is administered the composition by inhibiting the FXR receptor on L-cells.

Composition disclosed herein can be used for reducing HbA1C plasma levels in subjects. In some embodiments, dietary management of uncontrolled or sudden spikes in HbA1C plasma levels can also be controlled using the compositions disclosed here. The uncontrolled or spike in the HbA1C level might be due to insulin resistance, insulin insensitivity, Type-1-diabetes or Type-2-diabetes.

Compositions as disclosed herein can be also used for dietary management of gestational diabetes.

Compositions as disclosed herein can be used in treating type 2 diabetes (T2D) in rodent models. Studies done suggest the potential for individual microbial taxa in the human gut to improve or worsen metabolic disease via specific metabolites (signals) in hepatic or general circulation. In some embodiments, fecal and fasting circulating plasma metabolites measurement, implicated as mediators of microbiome effects, collected before and after the 12-week interventions showed some significant changes in the metabolites. In particular embodiments, there was increased colonic production of butyrate, paralleling changes in subject stool and production by in vitro culture. Some of the further changes include a decrease in medium chain fatty acids, their carnitinylates, and a decrease in bilirubin degradation byproducts when administered WBF-11 or WBF-10 compared to placebo.

In some embodiments, the compositions disclosed herein helps increase certain bile acids, especially ursodeoxycholic acid (UDCA). In vitro monoculture supernatant showed that the in some embodiments, in particular, C. butyricum strain specifically and efficiently synthesizes UDCA during butyrogenic growth in rich media amended with the primary bile acid chenodeoxycholic acid.

To our knowledge, this is the first description of an increase in circulating butyrate and UDCA due solely to a bacterial probiotic intervention in humans with T2D, and may help explain the observed improvement in postprandial glucose control.

In practicing the methods of treatment or use provided herein, therapeutically-effective amounts of the microbial compositions described herein are administered to a subject having a disease or condition to be treated. In some embodiments, the subject is a mammal such as a human. A therapeutically-effective amount can vary widely depending on the severity of the disease, the age and relative health of the subject, potency of the formulation, and other factors. Subjects can be, for example, humans, elderly adults, pregnant women, adults, adolescents, pre-adolescents, children, toddlers, infants, or neonates. A subject can be a patient. A subject can be an individual enrolled in a clinical study. A subject can be a laboratory animal, for example, a mammal, or a rodent.

In certain embodiments, the disclosure provides methods for the restoration of a microbial habitat of a subject to a healthy state. The method can comprise microbiome correction and/or adjustment including for example, replenishing native microbes, removing pathogenic microbes, administering prebiotics, and growth factors necessary for microbiome survival. In some embodiments, the method also comprises administering antimicrobial agents such as antibiotics.

The present disclosure provides methods for generalized-treatment recommendation for a subject as well as methods for subject-specific treatment recommendation. Such methods may be based on a microbiome profile of the subject. Methods for treatments can comprise one of the following steps: determining a first ratio of a level of a subject-specific microbiome profile to a level of a second microbiome profile in a biological sample obtained from at least one subject, detecting a presence or absence of a disease in the subject based upon the determining, and recommending to the subject at least one generalized or subject-specific treatment to ameliorate disease symptoms.

Health conditions that can be treated using the formulations described herein include, but are not limited to, inflammatory gut disorders like irritable bowel syndrome, inflammatory bowel disease, ulcerative colitis, diarrhea, constipation, leaky intestine, and/or Crohn's disease; liver disorders; biliary disorders; FXR-related cancers like breast cancer, liver cancer, colorectal cancer; gallbladder issues like gallstones; cholesterol metabolism disorders; or a combination thereof.

The present disclosure can provide for a diagnostic assay of at least one microbiome that includes a report that gives guidance on health status or treatment modalities for the health conditions described herein. The present disclosure can also provide therapeutic and/or cosmetic formulations for treatment of health conditions described herein.

The formulations described herein can be useful in the treatment and/or amelioration or management of specific symptoms. In embodiments, the formulations are used to reduce the size of gallstones or completely dissolve them in the gallbladder of the subject. In further embodiments, the formulations described herein are used to reduce pain in response to biliary distension or gallstones in a subject. In particular embodiments, the biliary distention is caused by obstruction to the flow of bile, with distension of the biliary lumen, and is clinically similar to when the obstruction occurs at the cystic duct or at another level of the common bile duct.

The formulations described herein can be useful in the treatment of FXR-related cancers. In particular embodiments, the formulation antagonizes the FXR-receptor on intestinal L cells.

The formulations described herein can be useful in the treatment of non-FXR-related cancers.

The formulations described herein can be useful in the treatment of a blood sugar disorder like insulin resistance, insulin insensitivity, Type-1 or Type-2 diabetes, or related disorders including obesity and/or cardiovascular disorders.

In some embodiments, the prebiotic and probiotic consortia are chosen to create an entirely self-sufficient system that does not require any external input. For example, a subject with an inflammatory gut disorder, biliary distension, liver disorders; cholesterol metabolism disorders; blood sugar disorders; or gallstones can be treated with a formulation of the disclosure which includes a prebiotic and possibly other agents.

Also provided are methods to generate probiotics against a subject's microbiome composition. The microbiome composition can have an effect on the subject's disease status and clinical treatment response. Compositions of the disclosure can be tailored to suit the microbiome composition of a subject for effective treatment of symptoms associated with a health condition. For example, therapeutic formulations for obese individuals can differ from therapeutic formulations for non-obese individuals for the treatment of a specific disorder based on differences in their microbiota.

A formulation can be administered by a suitable method for delivery to any part of the gastrointestinal tract of a subject including oral cavity, mouth, esophagus, stomach, duodenum, small intestine regions including duodenum, jejunum, ileum, and large intestine regions including cecum, colon, rectum, and anal canal. In some embodiments, the composition is formulated for delivery to the ileum and/or colon regions of the gastrointestinal tract. In some embodiments, the composition is formulated for delivery to the intestinal L-cells.

In some embodiments, administration of a formulation occurs orally, for example, through a capsule, pill, powder, tablet, gel, or liquid, designed to release the composition in the gastrointestinal tract. In some embodiments, administration of a formulation occurs by injection, for example, for a formulation comprising butyrate, propionate, acetate, and short-chain fatty acids. In some embodiments, the administration of a formulation occurs by application to the skin, for example, cream, liquid, or patch. In some embodiments, administration of a formulation occurs by a suppository and/or by enema. In some embodiments, a combination of administration routes is utilized.

In some embodiments, a formulation is administered before, during, and/or after treatment with an antimicrobial agent such as an antibiotic. For example, the formulation can be administered at least about 1 hour, 2 hours, 5 hours, 12 hours, 1 day, 3 days, 1 week, 2 weeks, 1 month, 6 months, or 1 year before and/or after treatment with an antibiotic. The formulation can be administered at most 1 hour, 2 hours, 5 hours, 12 hours, 1 day, 3 days, 1 week, 2 weeks, 1 month, 6 months, or 1 year before and/or after treatment with an antibiotic.

In some embodiments, the formulation is administered after treatment with an antibiotic. For example, the formulation can be administered after the entire antibiotic regimen or course is complete.

In some embodiments, a formulation is administered before, during, and/or after food intake by a subject. In some embodiments, the formulation is administered with food intake by the subject. In some embodiments, the formulation is administered with (e.g., simultaneously) with food intake.

In some embodiments, the formulation is administered before food intake by a subject. In some embodiments, the formulation is more effective or potent at treating a microbial condition when administered before food intake. For example, the formulation can be administered about 1 minute, about 2 minutes, about 3 minutes, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 12 hours, or about 1 day before food intake by a subject. For example, the formulation can be administered at least about 1 minute, about 2 minutes, about 3 minutes, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 12 hours, or about 1 day before food intake by a subject. For example, the formulation can be administered at most about 1 minute, about 2 minutes, about 3 minutes, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 12 hours, or about 1 day before food intake by a subject.

In some embodiments, the formulation is administered after food intake by the subject. In some embodiments, the formulation is more effective or potent at treating a microbial condition when administered after food intake. For example, the formulation can be administered at least about 1 minute, 2 minutes, 3 minutes, 5 minutes, 10 minutes, 15 minutes, 30 minutes, 45 minutes, 1 hour, 2 hours, 3 hours, 5 hours, 10 hours, 12 hours, or 1 day after food intake by a subject. For example, the formulation can be administered at most about 1 minute, 2 minutes, 3 minutes, 5 minutes, 10 minutes, 15 minutes, 30 minutes, 45 minutes, 1 hour, 2 hours, 3 hours, 5 hours, 10 hours, 12 hours, or 1 day after food intake by a subject.

Multiple therapeutic agents can be administered in any order or simultaneously. If simultaneously, the multiple therapeutic agents can be provided in a single, unified form, or in multiple forms, for example, as multiple separate pills. The composition can be packed together or separately, in a single package or in a plurality of packages. One or all of the therapeutic agents can be given in multiple doses. If not simultaneous, the timing between the multiple doses may vary to as much as about a month.

Compositions described herein can be administered before, during, or after the occurrence of a disease or condition, and the timing of administering the composition can vary. For example, the microbial composition can be used as a prophylactic and can be administered continuously to subjects with a propensity to conditions or diseases in order to lessen a likelihood of the occurrence of the disease or condition. The microbial compositions can be administered to a subject during or as soon as possible after the onset of the symptoms. The administration of the microbial compositions can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein. A composition can be administered as soon as is practicable after the onset of a disease or condition is detected or suspected, and for a length of time necessary for the treatment of the disease, such as, for example, from about 1 month to about 3 months. The length of treatment can vary for each subject.

Compositions described herein may be administered in combination with another therapy, for example, immunotherapy, chemotherapy, radiotherapy, anti-inflammatory agents, anti-viral agents, anti-microbial agents, and anti-fungal agents.

A composition of the disclosure can be administered in combination with another therapeutic agent for a metabolic or gut disorder. In some embodiments, a composition of the disclosure can be administered in combination with another therapeutic agent for irritable bowel syndrome, inflammatory bowel disease, ulcerative colitis, diarrhea, constipation, leaky intestine, and Crohn's disease. In some embodiments, a composition of the disclosure can be administered in combination with a therapeutic agent for irritable bowel syndrome. In some embodiments, the other therapeutic agent can serve as an adjuvant in modulating, potentiating, or boosting the effect of a composition of the disclosure in the subject.

Compositions described herein may be packaged as a kit. In some embodiments, a kit includes written instructions on the administration/use of the composition. The written material can be, for example, a label. The written material can suggest conditions and methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

### Dosing

The appropriate quantity of a therapeutic or cosmetic composition to be administered, the number of treatments, and unit dose can vary according to a subject and/or the disease state of the subject.

Compositions described herein can be in unit dosage forms suitable for single administration of precise dosages. In unit dosage form, the formulation can be divided into unit doses containing appropriate quantities of one or more microbial compositions. The unit dosage can be in the form of a package containing discrete quantities of the formulation. Non-limiting examples are liquids in vials or ampoules. Aqueous suspension compositions can be packaged in single-dose non-reclosable containers. The composition can be in a multi-dose format. Multiple-dose reclosable containers can be used, for example, in combination with a preservative. Formulations for parenteral injection can be presented in unit dosage form, for example, in ampoules, or in multi-dose containers with a preservative.

The dosage can be in the form of a solid, semi-solid, or liquid composition. Non-limiting examples of dosage forms suitable for use include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, dietary supplement, and any combination thereof.

A microbe can be present in any suitable concentration in a composition. The concentration of a microbe can be for example, from about 10¹ to about 10¹⁸ colony forming units (CFU). The concentration of a microbe can be, for example, at least 10¹, at least 10², at least 10³, at least 10⁴, at least 10⁵, at least 10⁶, at least 10⁷, at least 10⁸, at least 10⁹, at least 10¹⁰, at least 10¹¹, at least 10¹², at least 10¹³, at least 10¹⁴, at least 10¹⁵, at least 10¹⁶, at least 10¹⁷, or at least 10¹⁸CFU. The concentration of a microbe can be, for example, at most 10¹, at most 10², at most 10³, at most 10⁴, at most 10⁵, at most 10⁶, at most 10⁷, at most 10⁸, at most 10⁹, at most 10¹⁰, at most 10¹¹, at most 10¹², at most 10¹³, at most 10¹⁴, at most 10¹⁵, at most 10¹⁶, at most 10¹⁷, or at most 10¹⁸CFU. In some embodiments, the concentration of a microbe is from about 10⁸CFU to about 10⁹CFU. In some embodiments, the concentration of a microbe is about 10⁸CFU. In some embodiments, the concentration of a microbe is about 10⁹CFU.

Compositions as described herein may be formulated with any suitable therapeutically-effective concentration of prebiotic. For example, the therapeutically-effective concentration of a prebiotic can be at least about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. For example, the therapeutically-effective concentration of a prebiotic can be at most about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. For example, the therapeutically-effective concentration of a prebiotic can be about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. In some embodiments, the concentration of a prebiotic in a composition is about 70 mg/ml. In some embodiments, the prebiotic is inulin.

An effective amount of a composition can be administered in one or more administrations. Compositions as described herein may be administered, for example, 1, 2, 3, 4, 5, or more times daily. Compositions may be administered, for example, daily, every other day, three times a week, twice a week, once a week, or at other appropriate intervals for treatment of the condition.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Notwithstanding the appended claims, the present disclosure is also defined by the following embodiments:

1. A method of increasing ursodeoxycholic acid (UDCA) levels in a mammalian subject, the method comprising:
orally administering a composition comprising a bacterial strain selected from the group consisting of:
   a bacterial strain comprising a 16S rRNA having 95% or greater sequence identity to a 16S rRNA sequence of Akkermansia muciniphila;
   a bacterial strain comprising a 16S rRNA having 95% or greater sequence identity to a 16S rRNA sequence of Clostridium butyricum;
   a bacterial strain comprising a 16S rRNA having 95% or greater sequence identity to a 16S rRNA sequence of Anerobutyricum hallii;
   a bacterial strain comprising a 16S rRNA having 95% or greater sequence identity to a 16S rRNA sequence of Clostridium beijerinckii;
   a bacterial strain comprising a 16S rRNA having 95% or greater sequence identity to a 16S rRNA sequence of Bifidobacterium infantis; and
   any combination of bacterial strains thereof,
in an amount effective to increase UDCA levels in the mammalian subject.

2. A method of antagonizing farnesoid X receptor (FXR) in a mammalian subject, the method comprising:
orally administering a composition comprising a bacterial strain selected from the group consisting of:
   a bacterial strain comprising a 16S rRNA having 95% or greater sequence identity to a 16S rRNA sequence of Akkermansia muciniphila;
   a bacterial strain comprising a 16S rRNA having 95% or greater sequence identity to a 16S rRNA sequence of Clostridium butyricum;
   a bacterial strain comprising a 16S rRNA having 95% or greater sequence identity to a 16S rRNA sequence of Anerobutyricum hallii;
   a bacterial strain comprising a 16S rRNA having 95% or greater sequence identity to a 16S rRNA sequence of Clostridium beijerinckii;
   a bacterial strain comprising a 16S rRNA having 95% or greater sequence identity to a 16S rRNA sequence of Bifidobacterium infantis; and
   any combination of bacterial strains thereof,
in an amount effective to antagonize FXR in the mammalian subject.

3. The method according to embodiment 1 or embodiment 2, wherein the composition comprises a bacterial strain selected from the group consisting of: Akkermansia muciniphila, Clostridium butyricum, Anerobutyricum hallii, Clostridium beijerinckii, Bifidobacterium infantis, and any combination of bacterial strains thereof.

4. The method according to any one of embodiments 1 to 3, wherein the composition comprises two, three, four or each of the bacterial strains.

5. The method according to any one of embodiments 1 to 3, wherein the composition comprises bacterial strains consisting of Akkermansia muciniphila and Clostridium butyricum.

6. The method according to any one of embodiments 1 to 3, wherein the composition comprises a bacterial strain consisting of Clostridium butyricum.

7. The method according to any one of embodiments 1 to 6, wherein the composition further comprises a bacterial strain selected from the group consisting of a bacterial strain comprising a 16S rRNA having 95% or greater sequence identity to a 16S rRNA sequence of of Anaerostipes caccae, Bifidobacterium adolescentis, Bifidobacterium bifidum, Bifidobacterium longum, Butyrivibrio fibrisolvens, Clostridium acetobutylicum, Clostridium aminophilum, Clostridium colinum, Clostridium indolis, Clostridium orbiscindens, Enterococcus faecium, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faeccis, Roseburia hominis, Roseburia intestinalis, and any combination thereof.

8. The method according to any one of embodiments 1 to 7, wherein composition comprises 10\hat{ }8 or greater CFUs of each bacterial strain in the composition.

9. The method according to any one of embodiments 1 to 8, wherein the composition comprises a prebiotic.

10. The method according to embodiment 9, wherein the prebiotic is selected from the group consisting of: an amino acid, a peptide, biotin, polydextrose, a fructooligosaccharide (FOS), a galactooligosaccharide (GOS), a mannan oligosaccharide (MOS), a xylooligosaccharide (XOS), inulin, lignin, psyllium, chitin, chitosan, a gum, high amylose cornstarch (HAS), a β-glucan, lactulose, oligofructose-enriched inulin, oligofructose, oligodextrose, tagatose, trans-galactooligosaccharide, pectin, and any combination thereof.

11. The method according to embodiment 10, wherein the composition comprises inulin as a prebiotic.

12. The method according to any one of embodiments 1 to 11, wherein the composition comprises an enteric coating.

13. The method according to any one of embodiments 1 to 12, wherein the mammalian subject is human.

14. The method according to any one of embodiments 1 to 13, wherein the mammalian subject has diabetes.

15. The method according to embodiment 14, wherein the diabetes in type 2 diabetes.

16. The method according to any one of embodiments 1 to 15, wherein the mammalian subject suffers from a liver disorder, obesity, or both.

17. The method according to any one of embodiments 1 to 16, wherein the mammalian subject has colorectal cancer.

18. The method according to any one of embodiments 1 to 17, wherein the mammalian subject has gallstones.

19. The method according to any one of embodiments 1 to 18, wherein the mammalian subject has been instructed to not ingest a sulfonylurea (SFU) drug prior to the administering.

20. The method according to embodiment 19, wherein the SFU is selected from the group consisting of: glipizide, glimepiride, and glyburide.

21. A method of increasing ursodeoxycholic acid levels in a mammalian subject, the method comprising:
orally administering a composition comprising at least one microbe having a 95% sequence identity to a 16S rRNA sequence of Akkermansia muciniphila, Clostridium butyricum, Anerobutyricum hallii, Clostridium beijerinckii, and Bifidobacterium infantis to the mammalian subject;
thereby increasing ursodeoxycholic acid levels in the mammalian subject.

22. A method of increasing plasma levels of a ursodeoxycholic acid or a salt thereof in a mammalian subject, the method comprising:
orally administering a microbial composition comprising at least one microbe having a 95% sequence identity to a 16S rRNA sequence of Akkermansia muciniphila, Clostridium butyricum, Anerobutyricum hallii, Clostridium beijerinckii, and Bifidobacterium infantis to the mammalian subject,
wherein the microbial composition converts chenodeoxycholic acid into ursodeoxycholic acid or a salt thereof, thereby increasing plasma levels of the ursodeoxycholic acid or a salt thereof in the mammalian subject.

23. A method of antagonizing a FXR receptor, the method comprising:
orally administering a microbial composition comprising at least one microbe having a 95% sequence identity to a 16S rRNA sequence of Akkermansia muciniphila, Clostridium butyricum, Anerobutyricum hallii, Clostridium beijerinckii, and Bifidobacterium infantis to a mammalian subject; and
increasing UDCA levels in the mammalian subject, thereby antagonizing the FXR receptor by the increased UDCA in the mammalian subject.

24. A method of treating a disorder in a mammalian subject, the method comprising:
orally administering a composition comprising at least one microbe having a 95% sequence identity to a 16S rRNA sequence of Akkermansia muciniphila, Clostridium butyricum, anerobutyricum hallii, Clostridium beijerinckii, and Bifidobacterium infantis to the mammalian subject; and
antagonizing a FXR receptor in the mammalian subject, thereby treating the disorder in the mammalian subject,
wherein the disorder is selected from the group consisting of a liver disorder, a cholesterol metabolism disorder, an inflammatory gut disorder, obesity, dyslipidemia, and cancer.

25. The method according to any one of embodiments 21-24, wherein the composition comprises of Akkermansia muciniphila, Clostridium butyricum, Anerobutyricum hallii, Clostridium beijerinckii, and Bifidobacterium infantis.

26. The method according to any one of embodiments 21-25-23, wherein the composition further comprises a prebiotic.

27. The method according to embodiment 26, wherein the prebiotic is amino acid, a peptide, biotin, polydextrose, a fructooligosaccharide (FOS), a galactooligosaccharide (GOS), a mannan oligosaccharide (MOS), a xylooligosaccharide (XOS), inulin, lignin, psyllium, chitin, chitosan, a gum, high amylose cornstarch (HAS), a β-glucan, lactulose, oligofructose-enriched inulin, oligofructose, oligodextrose, tagatose, trans-galactooligosaccharide, pectin, and a combination thereof.

28. The method according to embodiment 26, wherein the prebiotic is inulin.

29. The method according to any one of embodiments 21-28, wherein the composition further comprises enteric coating or a preservative.

30. The method according to any one of embodiments 21-29, wherein the composition further comprises 97% sequence identity to an rRNA sequence from a microbe selected from the group consisting of Anaerostipes caccae, Bifidobacterium adolescentis, Bifidobacterium bifidum, Bifidobacterium longum, Butyrivibrio fibrisolvens, Clostridium acetobutylicum, Clostridium aminophilum, Clostridium colinum, Clostridium indolis, Clostridium orbiscindens, Enterococcus faecium, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faeccis, Roseburia hominis, Roseburia intestinalis, and any combination thereof.

31. The method according to any one of embodiments 21-30, wherein composition comprises at least 10\hat{ }8 CFUs of each of microbes in the composition.

32. The method of embodiment 21-31, wherein the subject suffers from a liver disorder, obesity or diabetes.

### EXAMPLES

Presented herein are analyses of the metabolite content of fasting plasma collected from participants at baseline, and at the end of the randomized intervention. These analyses include targeted measurements of short-chain fatty acids (SCFAs) and bile acids, as well as untargeted metabolomics. These observations were extended with experiments demonstrating in vitro biosynthesis by formulation strains of metabolites that appeared to increase among participants randomized to WBF-011. Also demonstrated is in vitro growth inhibition of some WBF-011 strains exposed to specific sulfonylurea drugs, the use of which appears to have attenuated glycemic improvement during the study.

SCFA, bile acid, and untargeted metabolomics analyses of circulating metabolites were performed from the plasma of human participants with T2D collected before and after a randomized, double-blind placebo controlled probiotic intervention that resulted in improved glucose control endpoints. The analyses revealed the following: (1) circulating butyrate significantly increased in the participants administered the 5-strain probiotic formulation; (2) circulating (G-)UDCA also significantly increased in these participants, representing a potential complementary or synergistic avenue for affecting the observed improvements; (3) one of the formulation strains, CBUT, performs the epimerization of CDCA to UDCA with a high conversion frequency; (4) untargeted metabolomics identified additional usage of SFU drugs by participants than previously understood, revealing a striking stratification of SFU-use and HbA1c non-response in the WBF-011 group only; (5) two of the three SFU drugs caused unambiguous growth inhibition of the probiotic strains during in vitro monoculture, representing a poorly explored potential side-effect for a drug class that is administered widely and is incompletely absorbed in the intestines.

The observed increases in circulating butyrate and UDCA following administration of a 5-strain probiotic that included a strain of Akkermansia represent potential synergistic mechanisms. Butyrate may also act synergistically, alongside FXR inhibition by UDCA, to further enhance GLP-1 secretion.

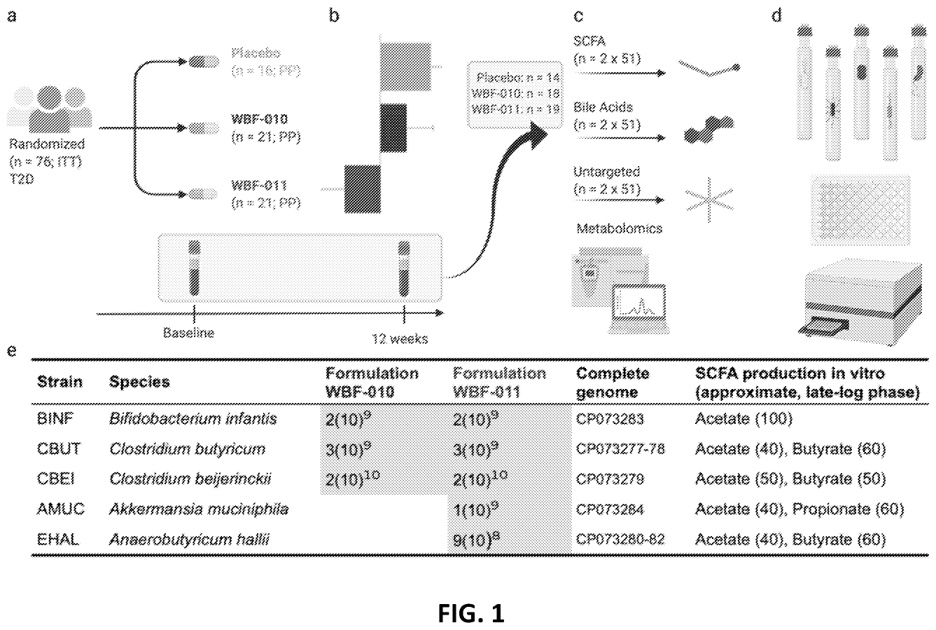

An outline of the provenance of the data in the present study is shown in FIG. 1. The clinical design is provided in FIG. 1a-b. Extended details into composition of the formulation are shown in FIG. 1e, including the accession numbers for the complete genome sequence of each strain and the typical ratios of major SCFAs produced by each strain after growth in rich media. A total of 76 participants with T2D were randomized as part of the intent-to-treat (ITT) cohort, with 58 successfully completing the study for inclusion in the per-protocol (PP) analysis (FIG. 1a-b). Collection of fasting blood plasma for exploratory analysis occurred at participant visits corresponding to baseline and cessation of intervention (˜12-weeks after baseline), resulting in 51 participants with successfully collected specimen pairs (and two collection duplicates) for a total of 104 specimens included in the main metabolomics collection. Additional details related to assay types, staging, and measurement vendors are summarized in Table 1.

TABLE 1
Summary of plasma metabolite specimens and measurements.

| | | | No. | No. | No. times |
|---|---|---|---|---|---|
| | | Source | Participants | Plasma | thawed |
| | | Clinical | (per | Speci- | prior to |
| Measurement | Vendor | Sites | protocol) | mens | measurement |
| Targeted [symbol] | [symbol] | [symbol] | 51 | 104 | 1 |
| Untargeted | [symbol] | [symbol] | 51 | 104 | 1 |
| Targeted [symbol] | [symbol] | [symbol] | 51 | 104 | 2 |
| Untargeted | [symbol] | [symbol] | [symbol] | [symbol] | 1 |

[symbol]

indicates data missing or illegible when filed

Measurements and replicate totals corresponding to experiments on formulation monocultures are summarized in Table 2.

TABLE 2
Summary of in vitro monoculture experiments.

| Measurement | Vendor | No. of Samples | Strains | Specimen |
|---|---|---|---|---|
| Untargeted | [symbol] | [symbol] | [symbol] | [symbol] |
| [symbol] | [symbol] | [symbol] | [symbol] | [symbol] |
| [symbol] | [symbol] | [symbol] | [symbol] | N/A |
| [symbol] | | [symbol] | | |

[symbol]

indicates data missing or illegible when filed

McMurdie et al. (2022) BMC Microbiology 22:19 is incorporated herein by reference in its entirety for all purposes.

### Example 1—Plasma Butyrate Increased Among the Major Short-Chain Fatty Acids, Correlated with Stool Butyrate and HbA1c Response

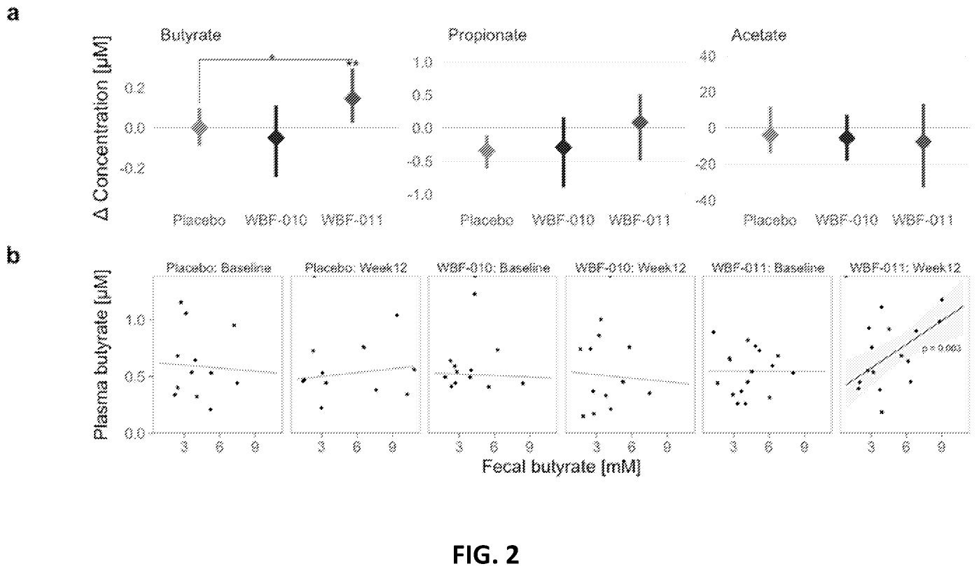

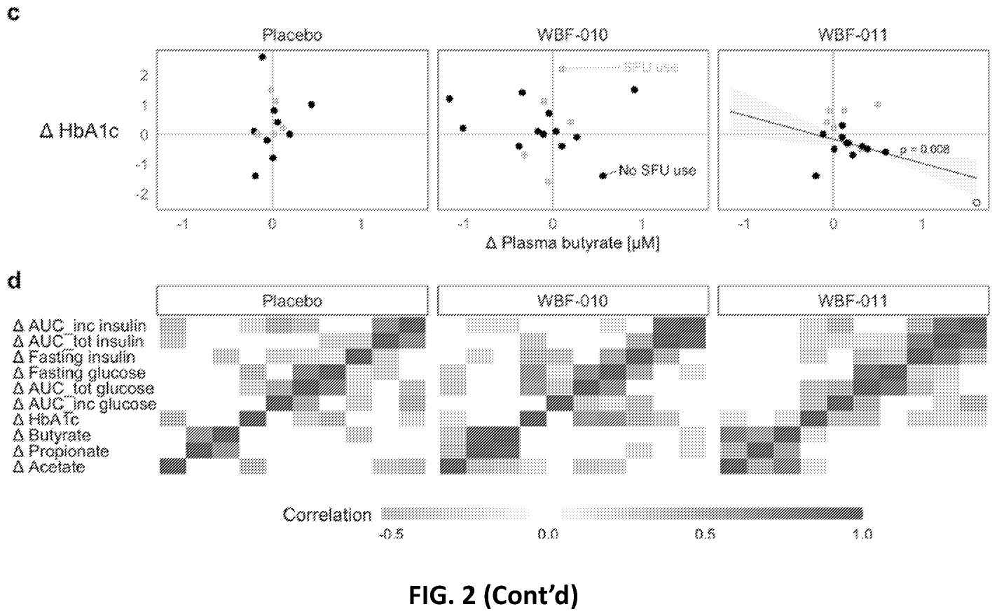

Among the three major gut-derived SCFAs (acetate, propionate, butyrate) only butyrate significantly increased (Wilcoxon within-group one-sided increase, p=0.007; WBF-011:placebo between-group one-sided increase, p<0.05), with a median increase in WBF-011 group 0.15 μM, or 27% increase from baseline after 12 weeks (FIG. 2a). Among the minor SCFAs measured, only valerate (C5) increased significantly relative to placebo (data not shown). The collection of stool and plasma from the same participant and clinical visit allowed for direct correspondence of the SCFAs of these specimen pairs. Among the SCFAs measured, robust linear regression detected a significantly non-zero slope only among the participants in the WBF-011 arm at the end of intervention (FIG. 2b, slope p=0.003). The glycated hemoglobin (HbA1c) value of study participants was also measured at these two clinical visits, and only in WBF-011 group we detected a significantly negative slope for participant-wise changes in HbA1c and plasma butyrate concentration (FIG. 2c, sulfonylurea non-users, slope p=0.008 with high leverage point omitted, p<10-6 if included; R2=0.85; Spearman's p<0.19), consistent with improvement in HbA1c with increasing butyrate. This negative correlation is shown in context with the correlation for other changes in SCFA and metabolic measures in FIG. 2d. That the detected butyrate was derived from the gut is canonical, particularly for plasma collected at the end of a fasting period, and supported by the aforementioned correlation with fecal butyrate of the WBF-011 group at the end of the 12-week intervention (FIG. 2b).

### Example 2—Plasma Ursodeoxycholate Increased Among the Bile Acids, Potentially Due to the Activity of C. butyricum (CBUT)

The results of independent detection and analysis of both untargeted and targeted bile acids for the main collection of 104 plasma samples is shown in FIG. 3a-b, respectively. With only a few exceptions, total bile acids appear to be within the nominal range (2-10 μM) for most participants and timepoints (FIG. 3c). For the untargeted measurements, secondary bile acids rank as the metabolite group (Metabolon “Sub-Pathway”) with the most significant non-zero median difference relative to placebo group (p=0.0056, FDR=0.12); and among these, gly-coursodeoxycholic acid (G-UDCA) and ursodeoxycholic acid (UDCA) had the smallest nominal p-values (respectively 0.0062, 0.0162). The targeted measures of bile acids also support a participant-wise increase in concentrations of G-UDCA and UDCA (FIG. 3b, +0.09 μM, nominal p=0.08).

In order to test the potential for individual strains to modify bile acids directly, in vitro monoculture experiments in growth media amended with primary bile acids were performed. An initial pilot experiment utilizing untargeted culture metabolomics implicated CBUT as the most-likely candidate for UDCA production (FIG. 3d), and this was followed by a targeted/quantitative assay for bile acids assay on fully replicated and controlled in vitro monoculture specimens (FIG. 3e). Only monoculture of CBUT amended with 50 μM of the human primary bile acid, CDCA, resulted in unambiguous synthesis of UDCA, with a conversion of 50-80% after 7 h incubation in rich media (FIG. 3e). As expected, no appreciable UDCA was detected in any conditions amended solely with 50 μM CA. The epimerization of CDCA to UDCA is canonically catalyzed via the action of two enzymes, 7α- and 7β-hydroxysteroid dehydrogenase (7αβ-HSDH). Of the five formulation strains, only the genome of CBUT is predicted to encode 7β-HSDH, and encodes the only 7α-HSDH with UniProt bitscore>200.

### Example 3—Additional Coordinated Changes Detected Via Untargeted Metabolomics

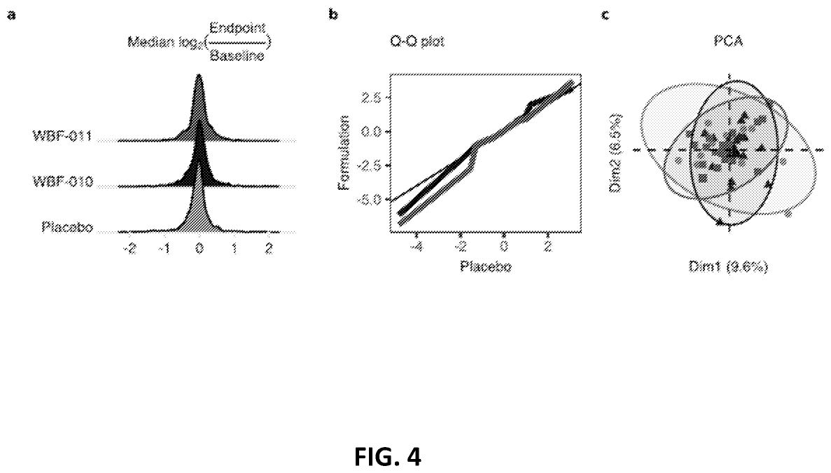

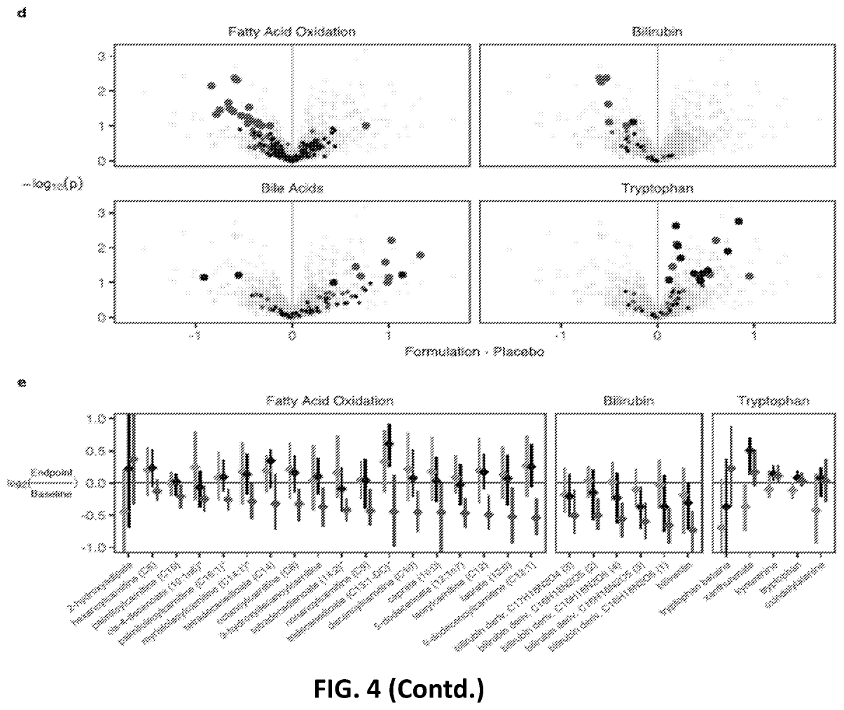

The initial untargeted survey of 1340 metabolites across 104 plasma specimens supports the null hypothesis that plasma metabolites did not change en masse within or between study arms (FIG. 4a-c), and no notable differences between the arms at baseline were detected. A multivariate summary of metabolite change values (participant-wise log ratio) via standard Principal Component Analysis indicates large overlap between the study arms (FIG. 4c) as well as a diffuse variance explained across many axes.

Multiple testing of within- and between-group changes for individual metabolites revealed a small number of metabolites that do appear to have changed with nominal statistical significance in the WBF-011 arm, and in a few cases these are within groups of interest for plasma metabolites and metabolic diseases (FIG. 4d-e). In particular, many metabolites associated with bilirubin degradation or medium chain acylcarnitines decreased, while a subset of bile acids (described above) and tryptophan intermediates increased. The complete tables of metabolite changes, including those not highlighted here or not associated with an a priori metabolite pathway group, are included in the reproducible data compendium corresponding to FIG. 4. The large number of untargeted metabolite features relative to the number of specimens/participants leads to a challenge to control false discoveries (Type-I error). This risk was mitigated by emphasizing groups of metabolites of prior interest that showed encouraging group-wise ranking and FDR. In the case of bile acids including ursodeoxycholate, the measurement in a targeted assay with a second metabolomics provider was also repeated. Aside from Type-I error, poorly annotatable metabolites as well as metabolites not associated with a larger coordinated group tended not to escape standard FDR correction even after independent filtering, and this arrangement may have inflated Type-II error due to incomplete knowledge about the identity or pathways of metabolite features, or their relevance.

Butyrate is shown to induce fatty acid oxidation, lipolysis, and thermogenesis, including in the liver and skeletal muscle. This is consistent the finding of a coordinated decrease in many acylcarnitines and their conjugate fatty acids in the WBF-011 group (‘Fatty Acid Oxidation’ group, FIG. 4d-e). The plasma profile of acylcarnitine esters provides a systemic snapshot of in vivo flux through specific steps of beta-oxidation, due especially to the regulated activity of carnitine acyltransferases that govern the exchange of CoA for carnitine to enable transport across the mitochondrial membrane. Adults that are obese or have T2D often exhibit elevated levels of acylcarnitines and free fatty acids in plasma, along with their conjugate fatty acids, derivatives of branched-chain and aromatic amino acids, and β-hydroxybutyrylcarnitine. No significant changes in the respective ensembles of branched-chain or aromatic amino acid plasma metabolites were detected.

### Example 4—Sulfonylurea Drug Use Stratifies Glucose Control Response

Three distinct sulfonylurea (SFU) drugs—glipizide, glimepiride, and glyburide—were detected in the plasma of some members of the study cohort. This includes a total of eight participants (placebo: 1, WBF-010: 3, WBF-011: 4) that had unambiguous levels of SFU drug at one or both collection events despite having no reported usage. The detection of SFU drugs in plasma through untargeted metabolomics appeared to be reliable. The detection of SFU drugs was unambiguous for SFU presence (ratio of signal to limit-of-detection>10 in the mildest case, >103 in most cases); there was perfect agreement (57/57) on SFU presence/absence between timepoints from the same participant; a majority of participants (15/19) showed agreement between timepoints regarding which SFU drug was detected (same molecule); and a majority of participants (recall, 11/13) recording SFU use had positive detection in their plasma. All SFU drugs were taken orally and absorbed in the intestines, with plasma half-lives (glipizide: 2-4 h, glimepiride: 5-8 h, glyburide: 4-10 h) and fecal elimination percentages (glipizide: 10%, glimepiride: 40%, glyburide: 50%) that vary by drug. These timescales and the consistency between timepoints indicate that the detection of SFU drugs in plasma was due to recent and likely ongoing use.

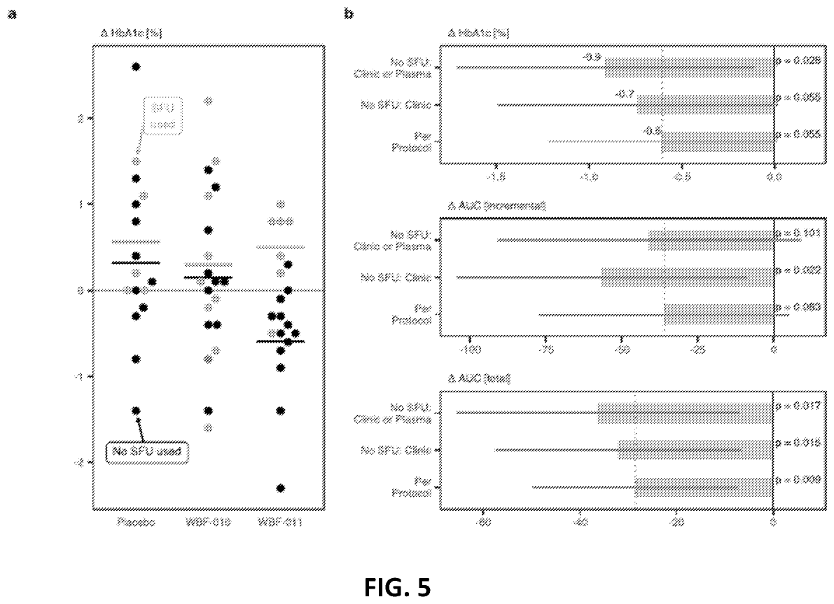

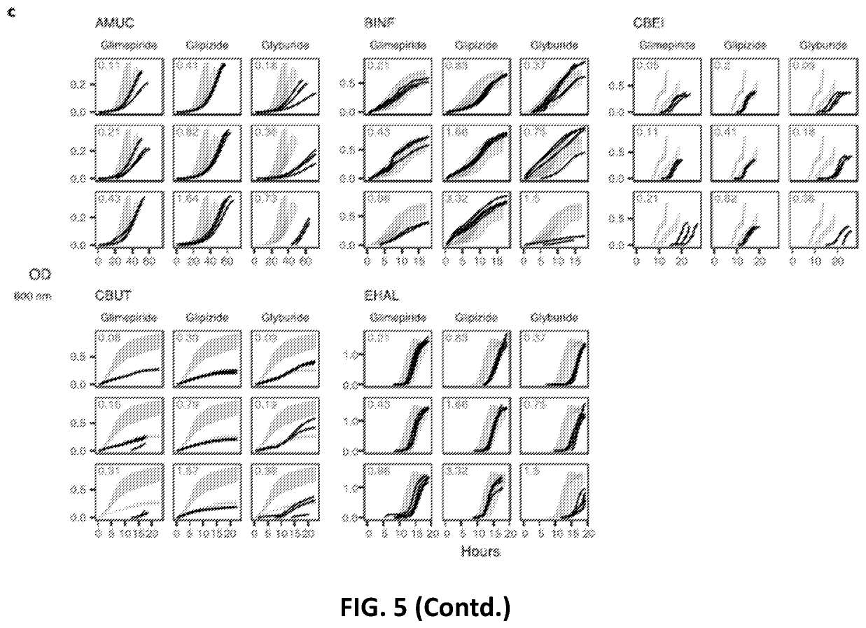

The additional participants determined through metabolomics to be using sulfonylurea drugs are overrepresented in the glycemically nonresponsive group, strengthening the post hoc endpoint stratification (FIG. 5a-b). Six of the seven participants with ΔHbA1c>0 (worse) in the WBF-011 group had detectable SFU drug, compared with three in the original clinical record. Placebo and WBF-010 groups showed only mild differences in ΔHbA1c between SFU users and non-users (FIG. 5a), such that the corresponding post hoc statistics for all glucose control endpoints improved for WBF-011 upon omission of SFU users; including a mean ΔHbA1c relative to placebo that improves from—0.6 originally to—0.9, with improved nominal significance (FIG. 5b).

Given SFU intestinal absorption and non-trivial fecal excretion, a priori, that direct inhibitory interaction may occur between a consumed SFU drug and the live strains of an orally administered probiotic, could not be excluded. In order to test whether SFU could directly inhibit the formulation strains, each was grown in monoculture in dilute rich media with or without the presence of each of the three detected SFUs. The SFU concentration was titrated in replicated conditions that spanned what might be expected in vivo according to typical daily dosing, average large intestinal volume, and solubility limits. Unambiguous concentration-dependent growth inhibition was observed (yield decrease, lag increase) for certain strains and SFUs (FIG. 5c). Glyburide and glimepiride exhibited the largest inhibitory effect overall, with patterns that were strain-specific. Importantly, glipizide was used by 5 of 7 participants using SFUs in the WBF-011 group and exhibited a substantially muted inhibitory effect under these conditions (FIG. 5c), complicating interpretation and suggesting additional or separate mechanisms may be required to explain the observed effect stratification.

### Materials and Methods for Examples 1-4

### Blood Plasma Sampling and Design

Adult human volunteer participants were considered eligible if they were diagnosed with T2D—fasting glucose≥126 mg/dL or HbA1c of ≥6.8%—with a body mass index between 25 and 45 kg/m², and treated with diet and exercise alone, or in combination with metformin with or without a sulfonylurea. Recruited participants were randomized to administration of one of three study formulations present in otherwise identical capsules, one of which contained only known inactive ingredients (placebo). Collection of fasting blood plasma for exploratory analysis occurred at a participant's baseline visit (prior to capsule administration) and at the designated visit completing the intervention period (the end of capsule administration) approximately 12-weeks after baseline. A total of 58 participants successfully completed the protocol and were included in final analyses. Each blood plasma specimen was derived from 10 mL of collected blood, which was allowed to clot for 30 min, and then spun at 1000-1300 xg for 20 min to collect the serum. Samples were maintained frozen (−20° C.) during on-premises storage at the clinic, then shipped on dry ice where they were stored at our facility at −80° C. prior to measurement at Metabolon, Inc. (Research Triangle Park, NC, USA) or MS-Omics (Vedbok, Denmark). The blood plasma specimens from all participants at one of the six study sites (four participants total) were excluded from the original metabolomics measurements due to a critical omission in dating the collected specimens. The fasting blood plasma from the remaining 51 study participants collected at the beginning and end of intervention, as well as two additional collection replicates (104 total specimens), were included in the main survey collection on which metabolite measurements were conducted (Table 1). All 104 plasma specimens were included in a targeted SCFA assay and an untargeted (metabolomics) assay conducted by Metabolon, as well as a targeted bile acids assay conducted by MS-Omics.

### Targeted SCFA Measurement, Metabolon

Targeted measurement survey of SCFA in the human plasma samples was accomplished via Ultrahigh Performance Liquid Chromatography-Tandem Mass Spectroscopy (Metabolon Method TAM148: “LC-MS/MS Method for the Quantitation of Short Chain Fatty Acid (C2 to C6) in Human Plasma and Serum”). The method utilizes a Waters ACQUITY ultra-performance liquid chromatography (UPLC) and a Thermo Scientific Q-Exactive high resolution/accurate mass spectrometer interfaced with a heated electrospray ionization (HESI-II) source and Orbitrap mass analyzer operated at 35,000 mass resolution. The following eight short or branched chain fatty acids were quantitated: acetic acid (C2), propionic acid (C3), isobutyric acid (C4, branched), butyric acid (C4), 2-methyl-butyric acid (C5, branched), isovaleric acid (C5, branched), valeric acid (C5), and hexanoic acid (C6). The human plasma samples were spiked with stable labelled internal standards, homogenized, and subjected to protein precipitation with an organic solvent. Following centrifugation, an aliquot of the supernatant was derivatized, and the reaction mixture then injected onto an Agilent 1290/AB Sciex QTrap 5500 LC MS/MS system equipped with a C18 reversed phase UHPLC column. The mass spectrometer was operated in negative mode using electrospray ionization. Quantitation was accomplished by adjusting the peak area of the individual analyte product ions with the peak area of the product ions of the corresponding internal standards, and via a weighted linear least squares regression analysis generated from fortified calibration standards prepared immediately prior to each run. Accuracy was evaluated using the corresponding QC replicates. LC-MS/MS raw data were collected and processed using SCIEX OS-MQ software v1.7.

### Untargeted Metabolomics Measurement, Metabolon

Untargeted metabolomics was generated by UHPLC-MS/MS, as above. Several recovery standards were added prior to the first step in the extraction process for quality control and standardization. Samples were then extracted with methanol under vigorous shaking for 2 min (Glen Mills GenoGrinder 2000) to precipitate protein and dissociate small molecules bound to protein or trapped in the precipitated protein matrix, followed by centrifugation to recover chemically diverse metabolites. Extracts are placed briefly on a TurboVap® (Zymark) to remove the organic solvent, and then stored overnight under nitrogen. The resulting extract was divided into the following five fractions: two for analysis by two separate reverse phase (RP)/UPLC-MS/MS methods using positive ion mode electrospray ionization (ESI), one for analysis by RP/UPLC-MS/MS using negative ion mode ESI, one for analysis by HILIC/UPLC-MS/MS using negative ion mode ESI, and one reserved for backup. The untargeted metabolomics platform identified 1340 metabolites in these same specimens with semi-quantitative precision that allows for relative comparison between samples, but not direct measures of concentration and with limited comparison between different metabolites. Note that untargeted metabolomics and targeted SCFA assays were conducted concurrently from separate aliquots during the same thawing event, such that the plasma samples were only thawed once at Metabolon facilities and otherwise maintained at cryogenic temperatures while in storage or in transit.

### Targeted Bile Acids Measurement, MS-Omics

A targeted bile acids assay was performed by MS-Omics on the remaining human plasma (twice-thawed prior to measurement) and in vitro culture specimens, for the quantitation of 18 of the most commonly encountered bile acids. Briefly, sample analysis was carried out using a Thermo Scientific Vanquish LC coupled to Thermo Q Exactive HF MS via electrospray ionization, performed in negative ionization mode. The chromatographic separation of bile acids was carried out on a Waters Acquity HSS T3 1.8 μm 2.1×150 mm (Waters). The column was thermostated at 30° C., with the mobile phases consisting of (A) ammonium acetate [10 mM], and (B) methanol:acetonitrile [1:1, v/v]. Bile acids were eluted by increasing B in A from 45 to 100% for 16 min, and a flow rate of 0.3 per minute. Peak areas were extracted using Tracefinder 4.1 (Thermo Scientific). Identification of compounds were based on mass and retention time of standards. An internal standard and a mixed pooled sample were analyzed at regular intervals for quality control.

### Untargeted Metabolomics Measurement, MS-Omics

An additional 20 plasma specimens from a sixth study site (8 specimens from per-protocol cohort, 12 additional specimens from participants that did not successfully complete the study) were collected (Table 1), but with a study event that was not adequately recorded to distinguish a participant's baseline and endpoint specimens. Untargeted metabolomics survey was performed on these samples by MS-Omics, with the primary goal of identifying whether sulfonylurea use could be detected or confirmed irrespective of study event. Briefly, the assay was carried out using a Thermo Scientific Vanquish LC coupled to Thermo Q Exactive HF MS with electrospray ionization interface. Analysis was performed in negative and positive ionization mode. Peak areas were extracted using Compound Discoverer 3.1 (Thermo Scientific). In general, the annotation of features as identifiable molecules occurred at four levels of decreasing qualitative confidence. The detection of sulfonylurea corresponded to ‘Level 2b’, identification by both accurate mass (with an accepted deviation of 3 ppm) and MS/MS spectra.

### In Vitro Bacterial Probiotic Strains Monoculture

In vitro monoculture experiments were conducted on formulation strains for metabolomics survey, determination of bile acid conversion, and evaluation of sensitivity to sulfonylurea drugs.

An initial pilot, untargeted metabolomics survey was conducted with each strain (n=1 per strain). Anaerobic growth was initiated on solid Peptone, Yeast Extract, Glucose medium (PYG, Anaerobe Systems ref. AS-8228) at 37° C. for 24-96 h in order to obtain single colonies. Single colonies were used to inoculate single, anoxic, 8 mL liquid hungate tubes containing PYG (Anaerobe Systems ref. AS-822) supplemented with 50 μM chenodeoxycholic acid (CDCA, VWR, ref. 10,003-180) and 50 μM cholic acid (CA, VWR, ref. AAA11257-14) from ethanol stocks (<1% ethanol final concentration). AMUC was grown in vegetable-based medium (VEG, 5.08 g/L NaCl, 0.4 g/L NaHCO₃, 0.04 g/L KH₂PO₄, 2.04 g/L K₂HPO₄, 0.02 g/L MgSO₄·7H₂O, 0.02 g/L CaCl₂), 2.5 g/L Na₂HPO₄, 0.5 g/L cysteine-HCl, 2 g/L dextrose, 2 g/L N-Acetylglucosamine, 7.5 g/L HiVeg™ Special Infusion, 10 g/L HiVeg™ Extract No. 2, 10 g/L HiVeg™ Peptone No. 3) without the amendment of bile acids due to preliminary data indicating this results in a notable growth lag and AMUC had limited genomic prediction for bile acids modification. Cultures were grown at 37° C., OD 600 nm was monitored and sampling was performed at mid to late log phase (OD range 2-5, depending on the strain). Each culture was centrifuged at 4° C. for 5 min at 5000 xg, the supernatant was filtered through 0.2 um pore size, and filtrate stored at −80° C. Cell pellets were washed twice in phosphate-buffered saline (PBS, ThermoFisher Scientific, 20,012,050), split into duplicate aliquots and stored at −80° C. Blank (uninoculated) media controls were prepared alongside cultures, as per the above protocol. All supernatants and cell pellets were shipped on dry ice to Metabolon for sample extraction and untargeted metabolomics analysis (as described above).

Culturing and sample preparation for analysis of bile acid transformation by WBF-011 strains was accomplished with growth conditions as above, with several modifications. Single colonies of CBUT, CBEI and EHAL were used to inoculate anoxic PYG medium, cultured to mid-log, and subsequently used to inoculate triplicate tubes, at an initial OD 600 nm of ˜0.05-0.1, for each of the two conditions: amended with 50 μM CA, amended with 50 μM CDCA. No-inoculum controls (blank media) were processed in triplicate, in parallel, for each condition. Initial samples (t₀) and final samples (t_{f}) were collected as above, without the washing of cell pellets in PBS. Wet weight for each cell pellet was recorded.

Growth sensitivity to SFU was performed in 96-well plates with 200 μL well volume, incubated within an anoxic chamber (Coy Laboratory Products). Each strain was separately cultivated in anoxic medium. CBEI and CBUT were cultivated in modified dilute PYG medium (mPYG, 0.08 g/L NaCl, 0.4 g/L NaHCO₃, 0.04 g/L KH₂PO₄, 0.02 g/L MgSO₄·7H₂O, 2.04 g/L K₂HPO₄, 0.02 g/L CaCl₂, 0.5 g/L cysteine-HCl, 5 g/L dextrose, 1 g/L yeast extract, 0.5 g/L HiVeg™ Peptone No. 1). AMUC was cultured in anoxic dilute vegetable-based medium (mVEG, 5.08 g/L NaCl, 0.4 g/L NaHCO₃, 0.04 g/L KH₂PO₄, 2.04 g/L K₂HPO₄, 0.02 g/L MgSO₄·7H₂O, 0.02 g/L CaCl₂, 2.5 g/L Na₂HPO₄, 0.5 g/L cysteine-HCl, 2 g/L dextrose, 4 g/L N-Acetylglucosamine, 1.19 g/L L-threonine, 1 g/L yeast extract, 1 g/L HiVeg™ Acid Hydrolysate). EHAL and BINF were cultivated in PYG (Anaerobe Systems ref. AS-8228). In ‘+BCAA’ conditions, the entire design of the SFU sensitivity was replicated, and branched chain amino acids (L-valine, L-leucine and L-isoleucine, Millipore Sigma, V0513, L8000 and 12752) were supplemented to a final concentration of 2.5 mM each. Anoxic SFU stocks of glyburide (Millipore Sigma, 356,310), glipizide (Millipore Sigma, G117-IG) and glimepiride (Millipore Sigma, G2295) were created at the indicated concentrations in DMSO and spiked into culture plates to achieve the final indicated SFU concentrations and DMSO percentages. The upper concentration for each SFU titration was initially selected based upon estimates for maximum in vivo concentrations assuming typical daily dosing and gut volume. These were later revised downward due to constraints arising from aqueous solubility and DMSO (vehicle) toxicity. Titrations were five-step two-fold dilution series from these upper concentrations. Culture plates were inoculated from frozen stocks of each strain and growth over 24-72 h was monitored via OD 600 nm, in an Epoch2 plate reader (Biotek Instruments, Inc). No-DMSO and no-SFU (DMSO-only) controls, as well as uninoculated controls, were included on each culture plate.

### Statistical Analysis and Graphics

As with all post hoc exploratory investigations, the methods and analyses presented here were not prescribed in the original study protocol nor Statistical Analysis Plan. Statistical significances are nevertheless provided as nominal ‘p values’ with comparison statistics, reflecting their value in description, disclosure, and ranking. All data analysis was performed using R version 4.0.2. Robust rank-based comparison (Wilcoxon Rank Sum) of within- and between-group differences was performed using the ‘wilcox.text’ of the ‘stats’ package in R. One- or two-sample and sidedness of tests are indicated with their respective description in Results and figure captions.

For consistency with the previous description of results, the comparison of within- and between-group changes in post hoc re-analysis of glucose control endpoints uses a standard Student's t-test (‘t.test’) as originally prescribed prior to unblinding in the 2018 statistical analysis plan for the study, NCT03893422. An exploratory multivariate summary of baseline or log-ratio metabolite change values used standard Principal Component Analysis (‘PCA’) implemented in the FactoMineR package. Robust regression wasperformed using the ‘robustbase’ package. The figures presented herein make extensive use of the ggplot2 package, with help from supporting packages ‘patchwork’, ‘ggbeeswarm’, ‘ggridges’, and ‘ggrepel’. Data processing, tidying, and joining was accomplished with help from ‘magrittr’ and ‘data.table’ packages. Growth curve data was smoothed via polynomial spline implemented in the pspline package

### Bacterial Genomic Analysis

Formulation strain genomes were sequenced using a PacBio Sequel and assembled using HiCan. To correct for sequencing errors in the PacBio assemblies, each genome was additionally sequenced using an Illumina Miseq sequencer and MiSeq Reagent Kit v3 (600-cycle, MS-102-3003) 2×300 bp paired-end mode. Library sizes were sufficient to achieve coverages of 500X, 250X, 250X and 200X for AMUC, EHAL, CBUT, and CBEI, respectively. The assemblies were corrected using Pilon and completeness in terms of circular contigs was checked using Circlator. The BINF genome was sequenced using PacBio RSII without additional short read sequencing. The query amino acid sequences of proteins involved in bile acid metabolism were obtained from Uniprot, limiting inclusion to Uniprot status ‘reviewed’. Candidate genes on formulation strain genomes were identified using Diamond and e-value maximum threshold of 10⁻¹⁰. Identification of an identical P9-encoding gene in strain AMUC (GenBank: CP073284, locus tag KDJ95_08250) was accomplished by mapping alignment of the sequence of the P9 gene (locus tag: Amuc_1631) at nucleotide positions 1,965,361 . . . 1967607 of Akkermansia muciniphila ATCC BAA-835 (GenBank: CP001071), with graphical inspection facilitated by Geneious Prime 2020.2.5.

### Example 5—Effect of Composition on a Subject with Gallstones

Patients with gallstones are treated with the compositions as shown in FIG. 1e, apart from the placebo group that was administered only inulin. After 12 weeks of administration of the WBF-11 composition, the gallstones disappear completely and/or reduce in size, compared to the placebo group which shows no significant changes.

### Example 6—Effect of Composition on a Subject with a Liver Disorder

Patients with NASH or NALFD are treated with the compositions as shown in FIG. 1e, apart from the placebo group that was administered only inulin. After 12 weeks of administration of the WBF-11 composition, fat accumulation in the liver shows significant improvement with reduced inflammation. The placebo group shows no significant changes.

### Example 7—Effect of Composition on Subject with Colorectal Cancer

Patients with a diagnosed colorectal cancer are treated with the compositions as shown in FIG. 1e, apart from the placebo group that was administered only inulin. After 12 weeks of administration of the WBF-11 composition, a reduction in the inflammation of the colon is observed. The placebo group shows no significant changes.

What is claimed is:

1. A method of increasing ursodeoxycholic acid (UDCA) levels in a mammalian subject, the method comprising:
orally administering a composition comprising a bacterial strain selected from the group consisting of:
a bacterial strain comprising a 16S rRNA having 95% or greater sequence identity to a 16S rRNA sequence of Akkermansia muciniphila;
a bacterial strain comprising a 16S rRNA having 95% or greater sequence identity to a 16S rRNA sequence of Clostridium butyricum;
a bacterial strain comprising a 16S rRNA having 95% or greater sequence identity to a 16S rRNA sequence of Anerobutyricum hallii;
a bacterial strain comprising a 16S rRNA having 95% or greater sequence identity to a 16S rRNA sequence of Clostridium beijerinckii;
a bacterial strain comprising a 16S rRNA having 95% or greater sequence identity to a 16S rRNA sequence of Bifidobacterium infantis; and
any combination of bacterial strains thereof,
in an amount effective to increase UDCA levels in the mammalian subject.

2. A method of antagonizing farnesoid X receptor (FXR) in a mammalian subject, the method comprising:
orally administering a composition comprising a bacterial strain selected from the group consisting of:
a bacterial strain comprising a 16S rRNA having 95% or greater sequence identity to a 16S rRNA sequence of Akkermansia muciniphila;
a bacterial strain comprising a 16S rRNA having 95% or greater sequence identity to a 16S rRNA sequence of Clostridium butyricum;
a bacterial strain comprising a 16S rRNA having 95% or greater sequence identity to a 16S rRNA sequence of Anerobutyricum hallii;
a bacterial strain comprising a 16S rRNA having 95% or greater sequence identity to a 16S rRNA sequence of Clostridium beijerinckii;
a bacterial strain comprising a 16S rRNA having 95% or greater sequence identity to a 16S rRNA sequence of Bifidobacterium infantis; and
any combination of bacterial strains thereof,
in an amount effective to antagonize FXR in the mammalian subject.

3. The method according to claim 1 or claim 2, wherein the composition comprises a bacterial strain selected from the group consisting of: Akkermansia muciniphila, Clostridium butyricum, Anerobutyricum hallii, Clostridium beijerinckii, Bifidobacterium infantis, and any combination of bacterial strains thereof.

4. The method according to any one of claims 1 to 3, wherein the composition comprises two, three, four or each of the bacterial strains.

5. The method according to any one of claims 1 to 3, wherein the composition comprises bacterial strains consisting of Akkermansia muciniphila and Clostridium butyricum.

6. The method according to any one of claims 1 to 3, wherein the composition comprises a bacterial strain consisting of Clostridium butyricum.

7. The method according to any one of claims 1 to 6, wherein the composition further comprises a bacterial strain selected from the group consisting of a bacterial strain comprising a 16S rRNA having 95% or greater sequence identity to a 16S rRNA sequence of of Anaerostipes caccae, Bifidobacterium adolescentis, Bifidobacterium bifidum, Bifidobacterium longum, Butyrivibrio fibrisolvens, Clostridium acetobutylicum, Clostridium aminophilum, Clostridium colinum, Clostridium indolis, Clostridium orbiscindens, Enterococcus faecium, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faeccis, Roseburia hominis, Roseburia intestinalis, and any combination thereof.

8. The method according to any one of claims 1 to 7, wherein composition comprises 10\hat{ }8 or greater CFUs of each bacterial strain in the composition.

9. The method according to any one of claims 1 to 8, wherein the composition comprises a prebiotic.

10. The method according to claim 9, wherein the prebiotic is selected from the group consisting of: an amino acid, a peptide, biotin, polydextrose, a fructooligosaccharide (FOS), a galactooligosaccharide (GOS), a mannan oligosaccharide (MOS), a xylooligosaccharide (XOS), inulin, lignin, psyllium, chitin, chitosan, a gum, high amylose cornstarch (HAS), a β-glucan, lactulose, oligofructose-enriched inulin, oligofructose, oligodextrose, tagatose, trans-galactooligosaccharide, pectin, and any combination thereof.

11. The method according to claim 10, wherein the composition comprises inulin as a prebiotic.

12. The method according to any one of claims 1 to 11, wherein the composition comprises an enteric coating.

13. The method according to any one of claims 1 to 12, wherein the mammalian subject is human.

14. The method according to any one of claims 1 to 13, wherein the mammalian subject has diabetes.

15. The method according to claim 14, wherein the diabetes in type 2 diabetes.

16. The method according to any one of claims 1 to 15, wherein the mammalian subject suffers from a liver disorder, obesity, or both.

17. The method according to any one of claims 1 to 16, wherein the mammalian subject has colorectal cancer.

18. The method according to any one of claims 1 to 17, wherein the mammalian subject has gallstones.

19. The method according to any one of claims 1 to 18, wherein the mammalian subject has been instructed to not ingest a sulfonylurea (SFU) drug prior to the administering.

20. The method according to claim 19, wherein the SFU is selected from the group consisting of: glipizide, glimepiride, and glyburide.
